(19) United States
(12) Patent Application Publication
(10) Pub. No.: US 2024/0164898 A1
(43) Pub. Date: May 23, 2024

(54) MECHANICALLY-EXPANDABLE PROSTHETIC HEART VALVE

(71) Applicant: EDWARDS LIFESCIENCES CORPORATION, Irvine, CA (US)

(72) Inventors: Noam Nir, Pardes-Hanna (IL); Michael Bukin, Pardes Hanna (IL); Eran Goldberg, Nesher (IL)

(21) Appl. No.: 18/430,453

(22) Filed: Feb. 1, 2024

Related U.S. Application Data
(63) Continuation of application No. PCT/US2022/037451, filed on Jul. 18, 2022.
(60) Provisional application No. 63/231,903, filed on Aug. 11, 2021.

(51) Int. Cl. A61F 2/24; A61B 17/00

(52) U.S. Cl. CPC A61F 2/243; A61B 17/00234; A61B 2017/00243; A61B 2017/0046; A61F 2220/0025

(57) ABSTRACT

A prosthetic heart valve includes an annular frame that is movable between a radially expanded state and a radially compressed state. The prosthetic heart valve includes one or more compliant actuators coupled to the annular frame. Each compliant actuator is configured to produce radial expansion of the annular frame and retain the annular frame in the radially expanded state. The prosthetic heart valve can be coupled to a handle of a delivery apparatus for delivery to an implantation site.

### CROSS-REFERENCE TO RELATED APPLICATIONS

This application is a continuation of International Application No. PCT/US2022/037451, filed Jul. 18, 2022, which claims the benefit of U.S. Provisional Application No. 63/231,903, filed Aug. 11, 2021, which is incorporated by reference herein in its entirety.

### FIELD

The disclosure relates to expandable prosthetic heart valves. The disclosure also relates to methods, assemblies, and apparatuses for delivering prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic heart valve can be mounted in a crimped (or compressed) state on the distal end of a delivery device and advanced through the patient's vasculature (for example, through a femoral artery and the aorta) until the prosthetic heart valve reaches the implantation site in the heart. The prosthetic heart valve is then expanded to its functional size. The prosthetic heart valve can be a mechanically expandable valve whose expansion relies on a mechanical actuator, a balloon-expandable valve whose expansion relies on an inflatable balloon, or a self-expanding valve that does not require an external force for expansion.

Mechanically expandable valves can include a plurality of expansion and locking assemblies that rely on discrete, noncompliant locking mechanisms, such as ratcheting mechanisms. Some expansion and locking assemblies can take up a relatively large amount of space (for example, by including a relatively large housing configured to accommodate an internal rack), which can impact the crimping profile of the valve.

Self-expanding valves have frames that are naturally biased to the radially expanded state. Such self-expanding valves can be held in a crimped state by a restraint during delivery to the implantation site and released from the restraint once at the implantation site, whereby the valves expand to their functional sizes. However, self-expanding valves are challenging to manufacture and deliver. For example, the entire frame is required to be operable within a wide range of diameters, and the forces exerted by the frame against the annulus must be high enough to retain the valve in the expanded state. Moreover, it is typically necessary to restrain the entire frame in the crimped state in a device (for example, a capsule) that extends along the entire length of the frame.

There is a need for a prosthetic heart valve that is relatively simple to manufacture and deliver and that is suitable for operation within a designated range of working diameters.

### SUMMARY

Disclosed herein are examples of compliant actuators that can be used to radially expand a prosthetic heart valve and maintain the prosthetic heart valve in a radially expanded state. The compliant actuators can be used alone or together with noncompliant actuators to move a prosthetic heart valve between a radially compressed state and a radially expanded state.

In a first representative example, a prosthetic heart valve comprises an annular frame, which comprises an inflow end, an outflow end, and a longitudinal axis extending from the inflow end to the outflow end and defining an axial direction. The annular frame is movable between a radially expanded state and a radially compressed state. The prosthetic heart valve further comprises at least one compliant actuator. The at least one compliant actuator comprises an extension spring that is coupled to the annular frame at first and second axially spaced locations. The extension spring is configured to resiliently urge the first and second locations toward each other to retain the annular frame in the radially expanded state against a radial compression force applied to the annular frame by tissue surrounding the prosthetic heart valve.

In a second representative example, a prosthetic heart valve comprises an annular frame, which in turn comprises an inflow end, an outflow end, and a longitudinal axis extending from the inflow end to the outflow end and defining an axial direction. The annular frame is movable between a radially expanded state and a radially compressed state. The prosthetic heart valve further comprises a plurality of compliant actuators positioned at spaced locations along a circumference of the annular frame. Each of the compliant actuators comprises a proximal connector head, a distal connector base, and at least one extension spring. The proximal connector head and the distal connector base are coupled to the annular frame at axially spaced locations. The at least one extension spring extends between and is attached at opposite ends to the proximal connector head and the distal connector base. The extension springs are configured to apply a radial expansion force to the annular frame.

In a third representative example, a prosthetic heart valve comprises an annular frame, which comprises an inflow end, an outflow end, and a longitudinal axis extending from the inflow end to the outflow end and defining an axial direction. The annular frame is movable between a radially expanded state and a radially compressed state. The prosthetic heart valve further comprises a compliant actuator. The compliant actuator comprises a proximal connector hub and a distal connector base coupled to the annular frame at axially spaced locations. The proximal connector hub includes a first lock arrangement, a locking head having a second lock arrangement complementary to the first lock arrangement, and a spring having a distal end attached to the distal connector base and a proximal end attached to the locking head. The locking head is movable axially relative to the proximal connector hub between a first position when the annular frame is in the radially compressed state and a second position when the annular frame is in the radially expanded state. When the locking head is moved to the second position, the second lock arrangement can engage the first lock arrangement to resist axial movement of the locking head relative to the proximal connector hub in a distal direction.

In a fourth representative example, a prosthetic heart valve comprises an annular frame, which comprises an inflow end, an outflow end, and a longitudinal axis extending from the inflow end to the outflow end and defining an axial direction. The annular frame is movable between a radially expanded state and a radially compressed state. The prosthetic heart valve further comprises a plurality of compliant actuators positioned at spaced locations along a circumference of the annular frame. Each of the compliant actuators comprises a proximal connector hub and a distal connector base coupled to the annular frame at axially spaced locations. The proximal connector hub includes a central opening and a first lock arrangement formed in the central opening; a locking head having a second lock arrangement complementary to the first lock arrangement; and a spring having a distal end attached to the distal connector base and a proximal end attached to the locking head. The locking head is movable from a first position between the proximal connector hub and the distal connector base in which the spring is in a relaxed state to a second position in which the second lock arrangement engages the first lock arrangement and the spring is in a loaded state.

In a fifth representative example, a prosthetic heart valve comprises an annular frame, which comprises an inflow end, an outflow end, and a longitudinal axis extending from the inflow end to the outflow end and defining an axial direction. The annular frame is movable between a radially expanded state and a radially compressed state. The prosthetic heart valve further comprises at least one compliant actuator coupled to the annular frame. The at least one compliant actuator is configured to produce radial expansion of the annular frame and retain the annular frame in the radially expanded state. The prosthetic heart valve further comprises at least one noncompliant actuator coupled to the annular frame. The at least one noncompliant actuator is configured to produce radial expansion and compression of the annular frame.

In a sixth representative example, a delivery assembly comprises a handle, at least one shaft extending distally from the handle, and a prosthetic heart valve according to any one of the first to the fifth representative example coupled to the at least one shaft.

In a seventh representative example, a method comprises radially compressing the annular frame of a prosthetic heart valve according to the second representative example by applying tension to the extension springs of the compliant actuators of the prosthetic heart valve. The method further comprises applying a restraining force to the annular frame to maintain the annular frame in the radially compressed state, inserting the prosthetic heart valve with the annular frame in the radially compressed state into a body of a patient, advancing the prosthetic heart valve with the annular frame in the radially compressed state to an implantation site within the body of the patient, radially expanding the annular frame by releasing the restraining force from the annular frame such that the extension springs revert to their respective free states, and retaining the annular frame in the radially expanded state by the compliant actuators.

In an eighth representative example, a method comprises inserting a prosthetic heart valve according to the third representative example into a body of a patient. The prosthetic heart valve is inserted with the annular frame in the radially compressed state and the spring of the compliant actuator of the prosthetic heart valve in the free state. The method further includes advancing the prosthetic heart valve to an implantation site within the body of the patient, axially displacing the locking head proximally relative to the distal connector base to extend the extension spring and position the locking head at the proximal connector hub, and retaining the locking head in the proximal connector hub by the first and second lock arrangements of the compliant actuator.

In a ninth representative example, a method of implanting a prosthetic heart valve within an anatomy comprises positioning a prosthetic heart valve in a radially compressed state within the anatomy and radially expanding the prosthetic heart valve to a first diameter at which an outward radial force produced by a compliant actuator coupled to a frame of the prosthetic heart valve is in a first equilibrium with a counter-force applied to the frame by the anatomy.

The foregoing general description and the following detailed description are exemplary of the invention and are intended to provide an overview or framework for understanding the nature of the invention as it is claimed. The accompanying drawings are included to provide further understanding of the invention and are incorporated in and constitute a part of the specification.

### BRIEF DESCRIPTION OF THE DRAWINGS

The following is a description of the figures in the accompanying drawings. In the drawings, identical reference numbers identify similar elements or acts.

FIG. 1 is a perspective view of a prosthetic heart valve, according to one example.

FIG. 2A is a side view of the frame of the prosthetic heart valve in a radially expanded state.

FIG. 2B is a side view of the frame of the prosthetic heart valve in a radially compressed state.

FIG. 3 is a perspective view of the frame of the prosthetic heart valve with compliant actuators, according to one example, configured to radially expand the frame and retain the frame in a radially expanded state.

FIG. 4A is a perspective view of the compliant actuator as depicted in FIG. 3.

FIG. 4B is a perspective view of a compliant actuator, according to another example.

FIG. 5A illustrates the compliant actuator as depicted in FIG. 4A mounted on a portion of the frame with the compliant actuator extending over two cells of the frame.

FIG. 5B illustrates multiple compliant actuators as depicted in FIG. 4A mounted on a portion of the frame with each compliant actuator extending over one cell of the frame.

FIG. 6A illustrates radial compression of the frame with the compliant actuator as depicted in FIG. 4A mounted to the frame.

FIG. 6B illustrates a radial component of a spring force applied by the compliant actuator to the radially compressed frame.

FIG. 6C illustrates radial expansion of the frame with the compliant actuator as depicted in FIG. 4A mounted to the frame.

FIG. 6D illustrates a radial component of a spring force applied by the compliant actuator to the radially expanded frame.

FIG. 7 is an elevated view of the frame in a radially compressed state with a restraining loop member extending around the frame.

FIG. 8 is a perspective view of a delivery assembly including a delivery apparatus and the prosthetic heart valve as depicted in FIG. 1.

FIG. 9 is a perspective view of a compliant actuator, according to another example.

FIG. 10 illustrates the compliant actuator as depicted in FIG. 9 mounted to a portion of the frame with the frame in a radially compressed state.

FIGS. 11A-11F illustrate various stages of transitioning the frame from a radially compressed state to a radially expanded state using the compliant actuator of FIG. 9.

FIG. 12 illustrates the compliant actuator as depicted in FIG. 9 mounted to a portion of the frame with the frame in a radially expanded state.

FIG. 13 is a perspective view of a proximal connector hub of the compliant actuator as depicted in FIG. 9.

FIG. 14 is a perspective view of a locking head of the compliant actuator as depicted in FIG. 9.

FIGS. 15A-15G illustrate various stages of fitting the locking head as depicted in FIG. 14 to the proximal connector hub as depicted in FIG. 13.

FIG. 16A is a side view of a compliant actuator, according to another example, including an alternative locking head and an alternative proximal connector hub.

FIG. 16B illustrates the locking head as depicted in FIG. 16A snapped to the respective proximal connector hub.

FIG. 16C illustrates a tension member attached between the locking head as depicted in FIG. 16A and the respective proximal connector hub.

FIG. 16D illustrates the tension member forming a rigid link between the locking head and the proximal connector hub.

FIG. 17 is a perspective view of a delivery assembly including a delivery apparatus and the prosthetic heart valve with the compliant actuator as depicted in FIG. 9.

### DETAILED DESCRIPTION

For the purposes of this description, certain specific details are set forth herein in order to provide a thorough understanding of disclosed examples. In some cases, as will be recognized by one skilled in the art, the disclosed examples may be practiced without one or more of these specific details, or may be practiced with other methods, structures, and materials not specifically disclosed herein. In some instances, well-known structures and/or processes associated with prosthetic heart valves and delivery apparatuses have been omitted to avoid obscuring novel and non-obvious aspects of the disclosed examples.

The disclosed examples are described with preferred implementations and examples. All the implementations and examples described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus.

Any of the prosthetic heart valves disclosed herein are adapted to be implanted in the native aortic annulus, although in other examples they can be adapted to be implanted in the other native annuluses of the heart (that is, the pulmonary, mitral, and tricuspid valves). The disclosed prosthetic heart valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic heart valves also can be implanted within a previously implanted prosthetic heart valve (which can be a prosthetic surgical valve or a prosthetic transcatheter heart valve) in a valve-in-valve procedure.

In some examples, the disclosed prosthetic heart valves can be implanted within a docking or anchoring device that is implanted within a native heart valve or a vessel. For instance, in one example, the disclosed prosthetic heart valves can be implanted within a docking device implanted within the pulmonary artery for replacing the function of a diseased pulmonary valve, such as disclosed in U.S. Patent Publication No. 2017/0231756, which is incorporated by reference herein. In another example, the disclosed prosthetic heart valves can be implanted within a docking device implanted within or at the native mitral valve, such as disclosed in International Patent Publication No. WO2020/247907, which is incorporated herein by reference. In another example, the disclosed prosthetic heart valves can be implanted within a docking device implanted within the superior or inferior vena cava for replacing the function of a diseased tricuspid valve, such as disclosed in U.S. Patent Publication No. 2019/0000615, which is incorporated herein by reference.

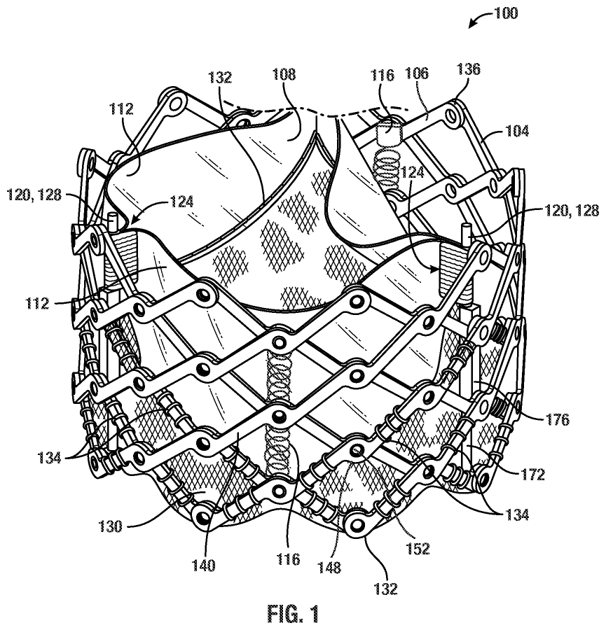

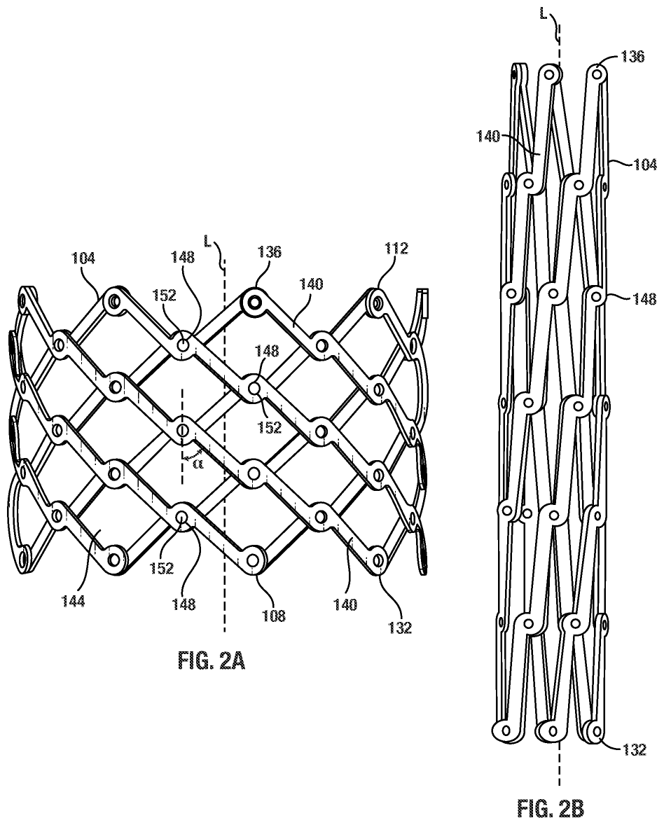

FIG. 1 illustrates an exemplary prosthetic heart valve 100, according to one implementation. The prosthetic heart valve 100 includes a mechanically expandable frame (or stent) 104 having an annular shape. The frame 104 has a longitudinal axis L (shown in FIG. 2A) that defines the axial direction of the prosthetic heart valve. The frame 104 includes a plurality of interconnected struts 140, which can be arranged in a lattice-type pattern. The struts 140 can pivot or bend to adjust the frame 104 between a radially compressed state and a radially expanded state (the radially expanded state is shown in FIG. 1).

The frame 104 includes an inflow end 132 and an outflow end 136, where the terms “inflow” and “outflow” are relative to the direction of blood flow through the prosthetic heart valve 100. Either of the inflow end 132 and the outflow end 136 can be proximal end of the prosthetic heart valve 100, depending on the technique used to deliver the prosthetic heart valve 100 to the implantation site. For example, in a transfemoral, retrograde delivery approach, the outflow end 136 can be the proximal end of the prosthetic heart valve. On the other hand, in trans-septal and transapical delivery approaches, the inflow end 132 can be the proximal end of the prosthetic heart valve.

The prosthetic heart valve 100 can include a plurality of compliant actuators 116 mounted to an inner surface 106 of the frame 104. The term “compliant” is used in the sense that the actuators 116 rely on elastic elements, such as springs. The compliant actuators 116 are configured to radially expand the frame 104 and, once the frame 104 is expanded, retain the frame 104 in the radially expanded state.

In some cases, as illustrated in FIG. 1, the prosthetic heart valve 100 optionally can further include a plurality of noncompliant actuators 120 mounted to the inner surface 106 of the frame 104. Unlike the compliant actuators 116, the noncompliant actuators 120 do not rely on elastic elements. The noncompliant actuators 120 can be configured to radially expand and/or radially compress the frame 104.

The noncompliant actuators 120 and compliant actuators 116 are spaced along a circumference of the frame 104. In one example, the noncompliant actuators 120 and compliant actuators 116 can be in an alternating arrangement along the circumference of the frame 104. The compliant actuators 116 and the noncompliant actuators 120 can be used at different stages of deploying the prosthetic heart valve 100 at an implantation site within a patient's body.

The prosthetic heart valve 100 can include a valvular structure 108 disposed within and coupled to the frame 104. The valvular structure 108 includes one or more leaflets 112 (three leaflets 112 are shown in FIG. 1 for illustrative purposes) that open and close to regulate flow of blood through the prosthetic heart valve 100. Each leaflet 112 can be made wholly or partly from biological material, bio-compatible synthetic materials, or other such materials. Suitable biological material can include, for example, bovine pericardium (or pericardium from other sources).

In one example, each leaflet 112 includes two opposing commissure tabs arranged on opposite sides of a body of the leaflet. The body of the leaflet may be the portion of the leaflet that is adapted to bend and move during operation of the prosthetic heart valve 100. The commissure tabs of adjacent leaflets 112 can be arranged to form commissures 124, which are coupled to commissure supports 128 mounted on the frame 104. In some examples, the commissure supports 128 can be support portions of the noncompliant actuators 120 (or support portions of the compliant actuators 116).

Further details regarding transcatheter prosthetic heart valves, including the manner in which the valvular structure can be mounted to the frame of the prosthetic heart valve can be found, for example, in U.S. Pat. Nos. 6,730,118, 7,393,360, 7,510,575, 7,993,394, and 8,252,202, U.S. Patent Publication Nos. 2018/0325665, 2019/0105153, and 2019/0192296, U.S. Patent Application No. 62/797,837, filed Jan. 28, 2019, 62/823,905, filed Mar. 26, 2019, 62/854,702, filed May 30, 2019, 62/928,993, filed Oct. 31, 2019, 62/959,723, filed Jan. 10, 2020, 62/971,011, filed Feb. 6, 2020, 62/985,558, filed Mar. 5, 2020, and 62/960,838, filed Jan. 14, 2020, and PCT Application Nos. PCT/US2019/61392, filed Nov. 14, 2019, PCT/US2020/18664, filed Feb. 18, 2020, all of which are incorporated herein by reference in their entireties.

The prosthetic heart valve 100 can include one or more skirts or sealing members. For example, the prosthetic heart valve 100 can include an inner skirt 130 mounted on the inner surface of the frame 104. In the example shown in FIG. 1, the inner skirt 130 is a circumferential inner skirt that spans an entire circumference of the inner surface of the frame 104. The inner skirt 130 can function as a sealing member to prevent or decrease perivalvular leakage (for example, when the prosthetic heart valve is placed at the implantation site) and as an attachment surface to anchor the leaflets 112 to the frame 104. For example, the inflow edges (for example, cusp) of the leaflets 112 can be sutured directly to the inner skirt 130 along a stitching line 132 (which can be referred to as a “scallop line”). The inner skirt 130 can be directly connected to selected struts 140 of the frame 104, such as with sutures 134.

In one implementation, the struts 140 can be pivoted to radially expand or radially compress the frame 104. As shown in FIG. 2A, the struts 140 are coupled together by pivot joints 148 that allow the struts 140 to pivot relative to each other. In one example, a pivot joint 148 between any two struts 140 can be formed by overlapping portions of the two struts and fastening the overlapping portions together by a pivot member, such as a rivet or pin 152. Each strut 140 can be connected to several other struts 140 at spaced locations by respective pivot joints 148. The inclination angle of the struts 140 (indicated as a in FIG. 2A) relative to the axial direction is greater when the frame 104 is radially expanded (shown in FIG. 2A) compared to when the frame 104 is radially compressed (shown in FIG. 2B).

In some examples, individual components of the frame 104 (for example, the struts 140 and rivets/pins 152) can be constructed individually and then mechanically assembled and coupled together. Further details regarding the construction of the frame are described in, for example, U.S. Patent Publication Nos. 2018/0153689, 2018/0344456, 2019/0060057, and 2019/0105153, and U.S. patent application Ser. No. 16/788,090, filed Feb. 11, 2020, and 62/945,000, filed Dec. 6, 2019, all of which are incorporated herein by reference.

In another implementation, the frame 104 can be formed as a unitary structure with non-pivoting joints between the struts of the frame. For example, the frame 104 can be formed (for example, via laser cutting, electroforming, or physical vapor deposition) from a single piece of material (for example, a metal tube). Examples of such frames are disclosed in U.S. Pat. No. 9,393,110 and U.S. Patent Publication No. 2018/0028310, which are incorporated herein by reference. The struts of the unitary structure can be formed so as to be bendable relative to each other to permit radial expansion and compression of the frame.

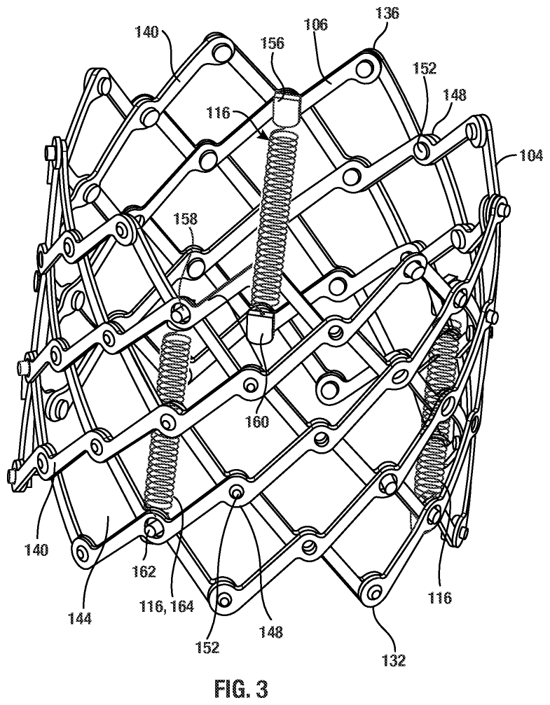

FIG. 3 shows a plurality of compliant actuators 116 mounted to the inner surface 106 of the frame 104. Three compliant actuators 116 are shown for illustrative purposes, although a greater or fewer number of actuators 116 can be used. In general, one or more compliant actuators 116 can be mounted to the frame 104. The compliant actuators 116 are spaced along the inner circumference of the frame 104. In some examples, the compliant actuators 116 can be evenly spaced along the inner circumference of the frame 104 to allow even distribution of radial forces exerted on the frame 104 by the compliant actuators 116.

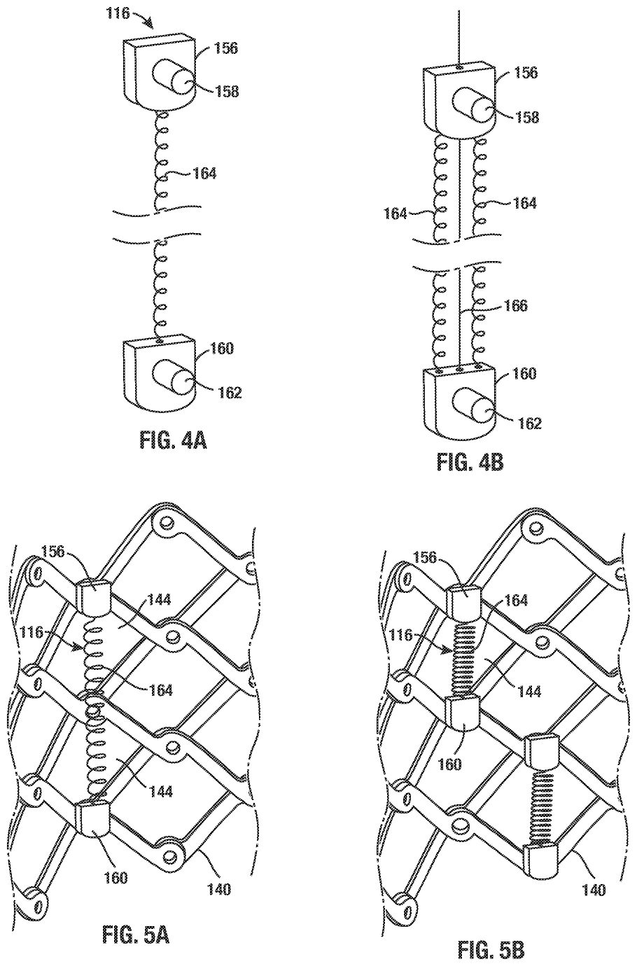

As shown more clearly in FIG. 4A, each compliant actuator 116 can include a proximal connector head 156, a distal connector base 160, and an elastic element or biasing element, such as a spring member 164. The proximal connector head 156 is proximal to the distal connector base 160 in the use position of the compliant actuator 116 on a frame. The spring member 164 extends between the proximal connector head 156 and the distal connector base 160 and is attached at the opposite ends to the proximal connector head 156 and the distal connector base 160. In another example, as illustrated in FIG. 4B, the elastic element includes two or more spring members 164 extending between and attached to the proximal connector head 156 and the distal connector base 160. In other examples, the elastic element can comprise an elastic cord or band made of any of various suitable elastomers.

In one example, each spring member 164 is an extension spring (or tension spring), which means that the spring member 164 is axially compressed (has an initial tension) in an unloaded position (or free state) and extends (stretches) in a loaded position (or tensioned state). In one example, the spring member 164 can be a helical or coil extension spring made of a biocompatible metal or alloy, such as stainless steel. The initial tension in the spring and the spring constant can be selected based on the range of working diameters of the prosthetic heart valve.

In some cases, the compliant actuator 116 can include a tension member attached to the distal connector base 160. As illustrated in FIG. 4B, for example, a tension member 166, such as a string or rod or cable, can be attached to the distal connector base 160 at one end. The other end of the tension member 166 can slide through a passage in the proximal connector head 156. Force can be applied to the tension member 166 to displace the distal connector base 160 relative to the proximal connector head 156.

In some examples, the proximal connector head 156 can include a pin member 158 or other suitable fastener to attach the proximal connector head to the frame 104. Similarly, the distal connector base 160 can include a pin member 162 or other suitable fastener to attach the distal connector base 160 to the frame 104. FIG. 3 shows the proximal connector head 156 and distal connector base 160 mounted to the frame 104 via the pin members 158, 162. In one example, each of the pin members 158, 162 can form part of one of the pivot joints 148 of the frame. That is, each of the pin members 158, 162 fastens overlapping portions of two struts together as previously described. In this case, the struts 140 connected to the pivot joints including the pin members 158, 162 will pivot relative to each other and relative to the proximal connector head 156 and distal connector base 160.

The proximal connector head 156 and distal connector base 160 of each compliant actuator 116 are mounted at axially spaced locations on the frame 104. Each compliant actuator 116 can extend over one cell 144 or two or more axially stacked cells 144 (that is, stacked in the axial direction) of the frame 104. FIG. 5A shows an example where the compliant actuator 116 extends over two axially stacked cells 144. FIG. 5B shows another example where the compliant actuator 116 extends over only one cell 144. FIG. 5B also demonstrates that the mounting positions of the proximal connector heads 156 relative to the proximal end (for example, the outflow end 136) of the prosthetic heart valve can vary along the circumference of the frame (that is, the compliant actuators 116 can be in a staggered arrangement along the circumference of the frame).

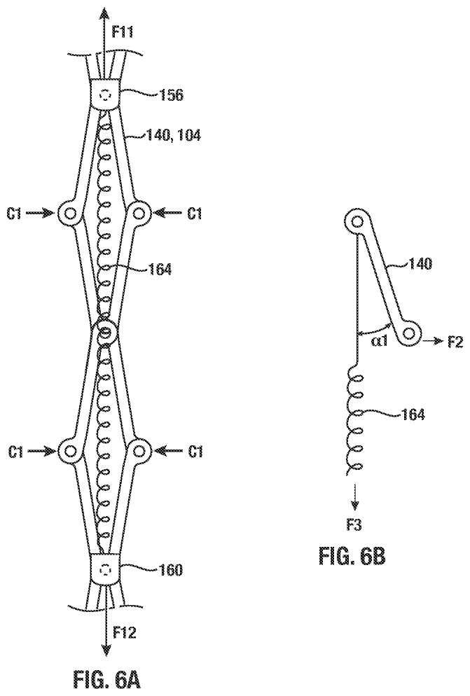

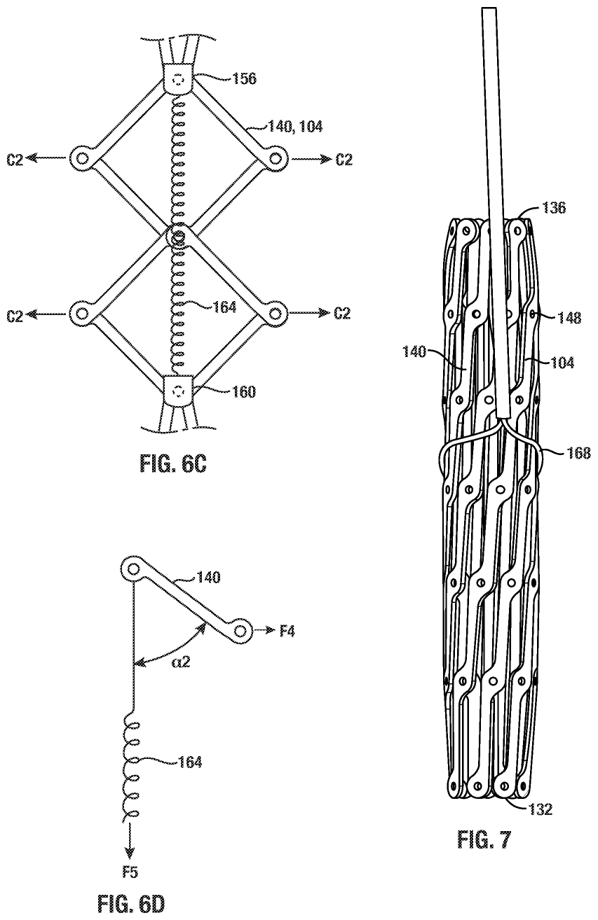

The spring members 164 can be tensioned (that is, stretched) by applying force to the junctions where the proximal connector heads 156 and distal connector bases 160 are attached to the frame 104. This loading of the spring members 164 results in pivoting or bending of the struts 140. For example, as illustrated in FIG. 6A, forces F11 and F12 can be applied to the junctions where the compliant actuator 116 is coupled to the frame 104 to pull the proximal connector head 156 and distal connector base 160 in a direction away from each other and generally parallel to the longitudinal axis of the frame, which would result in pivoting or bending of the struts 140 towards each other. This inward pivoting or bending of the struts 140 results in radial compression of the frame 104, as illustrated by arrows C1 in FIG. 6A.

FIG. 6B shows an angle α1 formed between the axial direction of the spring member 164 and the strut section 140 extending from the junction to which the spring member 164 is attached in a radially compressed state of the frame 104. The angle α1 is relatively small, which means that the force component F2 of the spring force F3 acting in the radial direction is also small.

Once the tension applied to the spring members 164 is removed, the spring members 164 revert to the free state. As the spring members 164 return to the free state, the proximal connector head 156 and distal connector base 160 move in a direction towards each other and generally parallel to the longitudinal axis of the frame 104. Simultaneously, the struts 140 pivot or bend away from each other, resulting in radial expansion of the frame 104, as illustrated by arrows C2 in FIG. 6C.

FIG. 6D shows an angle α2 between the axial direction of the spring member 164 and the strut section 140 extending from the junction to which the spring member 164 is attached in the radially expanded state of the frame 104. The angle α2 is relatively large when the frame 104 is in the radially expanded state, which means that the force component F4 of the spring force F5 acting in the radial direction is also large or relatively larger than spring force F2 (illustrated in FIG. 6B) when the frame is radially compressed. The angle α2 can be compared to the relatively small angle α1 shown in FIG. 6B for the radially compressed state of the frame.

The force component F4 represents the radial force that acts to maintain the frame 104 in a radially expanded state. In one example, each compliant actuator 116 can be configured to exert a radial force in a range of about 20N to 40N when the frame 104 is in the radially expanded state. The actuators 116 can exert a total force of about 60N to 120N. In some examples, the compliant actuators 116 configured to exert a radial force in this range can be used with working diameters in a range from 26 mm to 29 mm (working diameter is the diameter of the frame when radially expanded to the use position at the implantation site). The compliant actuators 116 can be configured by setting the proper dimensions and material properties of the spring members 164 to arrive at a desired spring coefficient for the expected range of radial forces and angles α2.

The frame 104 with the compliant actuators 116 behaves like a self-expanding valve. For delivery, the frame 104 can be radially compressed to position the prosthetic heart valve in a crimped state. At the crimped state, each of the spring members 164 exerts its maximum force and tries to expand the frame 104. Therefore, the frame must be restrained to prevent the frame from expanding prematurely. In one example, the frame can be restrained by confining the frame (with the spring members in the tensioned state) in a capsule or sheath of a delivery apparatus. Removal of the capsule from the frame will allow the spring members 164 to move toward the free state and expand the prosthetic valve.

When the prosthetic valve is in its fully expanded state, the spring members 164 desirably are still in a tensioned state so as to maintain a constant expansion force on the frame 104. In another implementation, the spring members 164 can be configured to attain their free state (fully axially compressed) when the prosthetic valve is in its fully expanded state and the resistance of the spring members 164 against axial elongation is sufficient to resist radial compression of the prosthetic valve under forces from the surrounding anatomy.

In another example, as illustrated in FIG. 7, a loop member 168 (for example, wire, cable, suture, and the like) can be the restraining member. As shown in FIG. 7, the loop member 168 extends around the frame 104 in the form of a lasso or adjustable loop. With the spring members 164 in a tensioned state, the loop member 168 can be tensioned or tightened around the frame 104 to retain the frame 104 in the radially compressed state. Subsequent release of tension from the loop member 168 will allow the spring members 164 to return to the free state. The loop member can also be a band that can be slipped over the radially compressed frame and torn or otherwise removed when radial expansion of the frame 104 is desired. The loop member as a restraining device can have the advantage of simplicity over a full capsule.

Returning to FIG. 1, the prosthetic heart valve 100 can include only the compliant actuators 116 or can include both the compliant actuators 116 and the noncompliant actuators 120. Whereas the compliant actuators 116 can radially expand the frame 104 and retain the frame 104 in the radially expanded state, the noncompliant actuators 120 can be configured to radially expand and compress the frame 104. In one example, the compliant actuators 116 can be relied on to expand the frame 104 and retain the frame 104 in the expanded state. If additional radial force is required to fully expand the prosthetic heart valve, the noncompliant actuators 120 can provide the additional radial force. In lieu of or in addition to using the noncompliant actuators 120 to provide additional radial force to expand the prosthetic valve, the tension members 166 (shown in FIG. 4B) of the compliant actuators 116 can be used to assist in radially expanding the frame 104, whereafter the compliant actuators 116 will maintain the frame in the radially expanded state. If after expanding the prosthetic heart valve it is desired to recompress the prosthetic heart valve, the noncompliant actuators 120 may be used to recompress the prosthetic heart valve.

In one example, the noncompliant actuators 120 are linear actuators with axially movable members. For example, each actuator 120 can include an inner member 172 (or piston) and an outer member 176 (or cylinder) that are movable relative to each other in the axial direction of the prosthetic heart valve. The inner member 172 is pivotably coupled to a junction of the frame 104, such as a junction at the inflow end 132, while the outer member 176 is pivotably coupled to another junction of the frame 104 closer to the outflow end 136. Relative movement between the inner member 172 and outer member 176 radially expands or compresses the prosthetic heart valve. For example, movement of the inner member 172 in a direction towards the inflow end 132 and/or movement of the outer member 176 in a direction towards the outflow end 136 can radially compress the prosthetic heart valve. Conversely, movement of the inner member 172 in a direction towards the outflow end 136 and/or movement of the outer member 176 in a direction towards the inflow end 132 can radially expand the prosthetic heart valve.

In another example, the noncompliant actuators 120 can be pull members, such as in the form of sutures, tethers, cables, wires, rods, and the like that have distal ends connected to junctions of the frame. The pull members are configured to apply a proximally directed force to the frame, which in conjunction with a distally directed force applied to the frame by a delivery apparatus cause the frame to expand radially. Further details regarding noncompliant actuators that can be implemented in the presently disclosed prosthetic valves are provided in U.S. Publication No. 2018/0153689.

Advantageously, the compliant actuators 116 allow a mechanically expandable prosthetic heart valve to be radially expanded and retained in a radially expanded state without separate components for locking the valve in the radially expanded state.

In some cases, the radial force required to expand a calcified native annulus during a transcatheter aortic valve replacement (TAVR) procedure may be significantly greater than the radial force required to hold the prosthetic valve in a functional expanded state. Advantageously, the compliant actuators 116 can be used with mechanical actuation mechanisms (for example, the noncompliant actuators 120, or other actuation mechanisms such as pull members). The mechanical actuation mechanisms can be operated to apply additional expansion forces to the prosthetic heart valve as needed to achieve a desired working diameter. After expanding the prosthetic heart valve, the compliant actuators 116 can retain the prosthetic heart valve at the working diameter without further aid of the mechanical actuation mechanisms.

Since the radial force exerted by the compliant actuators 116 in the radially compressed state of the prosthetic heart valve is relatively small, the prosthetic heart valve can be effectively retained in a crimped state using a loop member (instead of a capsule spanning the entire length of the valve), which can allow a simplified delivery assembly.

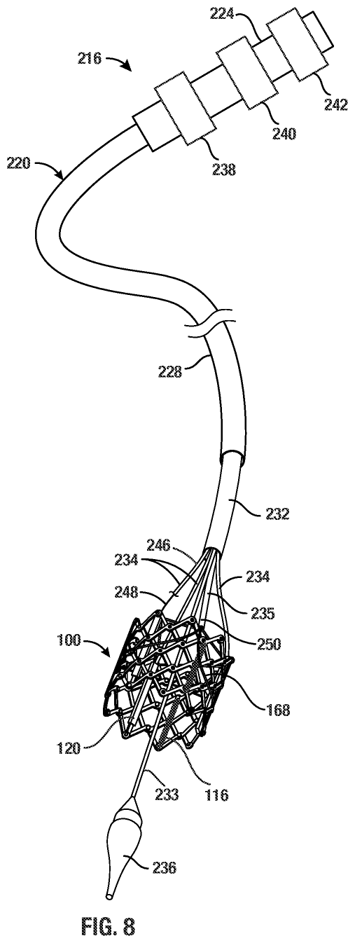

FIG. 8 shows an exemplary delivery assembly 216 including a delivery apparatus 220 and the prosthetic heart valve 100. For ease of illustration, some details of the prosthetic heart valve 100 are omitted from FIG. 8. However, these details can be found in FIGS. 1-7. In the example of FIG. 8, the prosthetic heart valve 100 includes a plurality of compliant actuators 116 and a plurality of noncompliant actuators 120, as previously described. The prosthetic heart valve 100 is shown in the radially expanded state in FIG. 8. In general, the prosthetic heart valve 100 will be positioned in a crimped state prior to delivery to an implantation site within a patient's body.

The delivery apparatus 220 can generally include a handle 224, a first shaft (an outer shaft in the illustrated example) 228 extending distally from the handle 224, and a second shaft (an intermediate shaft in the illustrated example) 232 extending through the first shaft 228, and a third shaft 233 (an inner shaft in the illustrated example) extending through the second shaft 232. In some cases, the second shaft 232 can be a multi-lumen shaft. The prosthetic heart valve 100 can be disposed around a distal portion of the third shaft 233. A nosecone 236 can be mounted to the distal end of the third shaft 233. The third shaft 233 and the nosecone 236 can define a guidewire lumen sized for receiving a guidewire so that the delivery apparatus 220 can be advanced over a guidewire previously inserted into a patient's body.

In some cases, one or more actuation assemblies 234 can extend through lumens of the second shaft 232 or can be otherwise coupled to the second shaft 232. The prosthetic heart valve 100 can be releasably coupled to the second shaft 232, for example, via the actuation assemblies 234. In some cases, the actuation assemblies 234 can operate the actuators 120. For example, each actuation assembly 234 can include an outer member 246 and an inner member 248 extending through the outer member 246. The inner member 248 of each actuation assembly 234 can be releasably connected to an inner member 172 of a corresponding actuator 120 of the prosthetic valve and the outer member 246 of each actuation assembly 234 can abut an outer member 176 of the corresponding actuator 120 or a location on the frame 104. In this manner, axial movement of the inner member 248 relative to the outer member 246 is transferred to the inner member 172 of the actuator 120, producing radial expansion of the frame 104. The actuation assemblies 234 can be disengaged from the actuators 120 after the prosthetic heart valve 100 has been deployed and secured against the native annulus.

In some cases, the delivery apparatus can include a valve-restraining device 235 configured to retain the prosthetic heart valve 100 in a radially compressed state during delivery through a patient's vasculature and control expansion of the prosthetic valve at or near the target implantation site. The valve-restraining device 235 can comprise a shaft 250 and the adjustable loop member 168 extending from the distal end of the shaft 250. The shaft 250 can have a proximal end connected to the handle 224 and can extend distally from the handle through the second shaft 232. The loop member 168 can be a loop formed along a distal end portion of an elongated tether that extends through the shaft 250. The free ends of the tether can be operatively coupled to the handle 224. The size of the loop member 168 encircling the prosthetic heart valve 100 can be adjusted by relative movement between the shaft 250 and the tether. For example, pulling the tether proximally through the shaft 250 is effective constrict the loop member 168 and radially compress the prosthetic valve while introducing slack in the tether allows the loop member 168 to increase in diameter, thereby allowing the prosthetic valve to expand. Further details of the valve-restraining device 235 are disclosed in U.S. Publication No. 2020/0188099, which is incorporated herein by reference. The valve-restraining device 235 can be referred to as a “recompression device” because it can be used to recompress the prosthetic valve after it has been expanded inside the patient's body.

The handle 224 of the delivery apparatus 220 can include one or more control mechanisms (for example, knobs or other actuating mechanisms) for controlling different components of the delivery apparatus 220 to steer the delivery apparatus and/or to expand and/or deploy the prosthetic heart valve 100 at an implantation site. For example, as illustrated in FIG. 8, the handle 224 can include first, second, and third knobs 238, 240, and 242.

The first knob 238 can be a rotatable knob configured as a steering mechanism that can adjust the curvature of the first shaft 228. The first knob 238 can be operatively coupled to a pull member (for example, a pull wire) that extends through a lumen of the outer shaft and has a distal end affixed to the outer shaft. Rotation of the first knob 238 is effective to control the tension in the pull member, which in turn adjusts the curvature of a distal end portion of the first shaft 228 for steering the delivery apparatus as it is advanced through the patient's vasculature. Further details of the steering mechanism and pull member are disclosed in U.S. Publication No. 2013/0030519, which is incorporated herein by reference.

The second knob 240 can be a rotatable knob configured to produce radial expansion and/or contraction of the prosthetic heart valve 100. Rotation of the second knob 240 in a first direction (for example, clockwise) can radially expand the prosthetic heart valve 100 and rotation of the second knob 240 in a second direction (for example, counter-clockwise) can radially collapse the prosthetic heart valve 100. In other examples, the second knob 240 can be actuated by sliding or moving the knob 240 axially, such as pulling and/or pushing the knob. The operation to be carried out when the second knob 240 is actuated will depend on the configuration of the actuators mounted to the frame of the prosthetic heart valve. In one implementation, for example, actuation of the second knob 240 can increase the diameter of the loop member 168, such as by introducing slack in the tether forming the loop member 168. This allows the prosthetic heart valve 100 to expand under the expansion force exerted on the frame by the compliant actuators 116. If desired, after the prosthetic valve is expanded, the size of the loop member 168 can be reduced to recompress the prosthetic valve for repositioning the prosthetic valve and/or removing the prosthetic valve from the patient's body.

The third knob 242 can be used to actuate the actuators 120 of the prosthetic valve (when present) via the actuation assemblies 234 to assist in the expansion of the prosthetic valve in conjunction with the compliant actuators 116. For example, rotation of the knob 242 in a first direction can move the inner members 248 proximally relative to the outer members 246, which in turn moves the inner members 172 of the actuators 120 relative to the outer members 176 of the actuators 120, further expanding the prosthetic valve. Rotation of the knob 242 in a second direction can move the inner members 248 distally relative to the outer members 246 to assist in recompressing the prosthetic valve. As noted above, in some examples, the prosthetic heart valve 100 need not include any actuators 120 and instead can rely solely on the compliant actuators 116 for full radial expansion of the prosthetic valve. In such examples, the actuation assemblies 234 can be replaced with valve-connection devices that form a releasable connection with the prosthetic valve (without providing any valve expansion function) and the knob 242 can be configured to disengage the valve-connection devices from the prosthetic valve. In other examples, the third knob 242 can be actuated by sliding or moving the third knob 242 axially, such as pulling and/or pushing the knob.

In examples where the delivery apparatus 220 includes the actuation assemblies 234 for valve expansion, a fourth knob (not shown) can be provided to disconnect the actuation assemblies 234 from the linear actuators 120, thereby releasing the prosthetic valve from the delivery apparatus. The fourth knob can be a rotatable knob operatively connected to the actuation assemblies such that rotation of the fourth knob decouples the actuation assemblies from the prosthetic heart valve 100. In other examples, the fourth knob can be actuated by sliding or moving the fourth knob axially, such as pulling and/or pushing the knob.

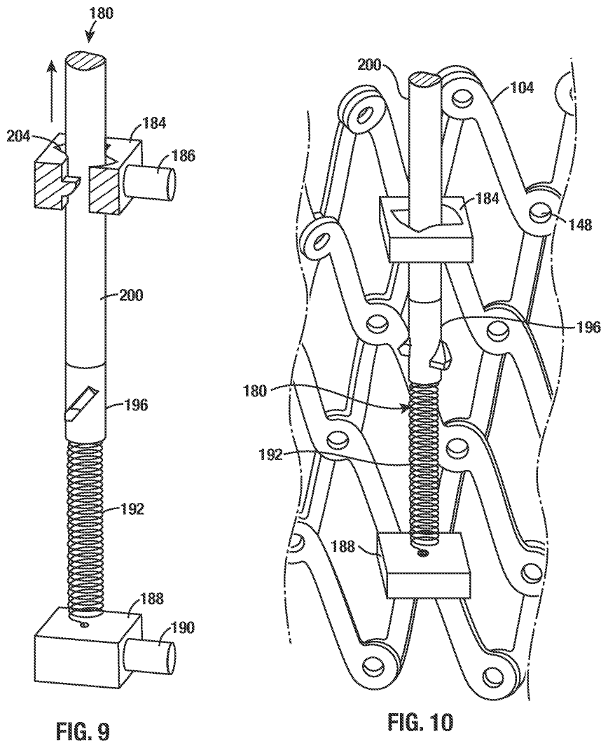

FIG. 9 shows a compliant actuator 180, according to another implementation. The compliant actuator 180 is configured to radially expand the frame 104 and retain the frame 104 in the radially expanded state. Multiple compliant actuators 180 can be mounted at spaced positions along the inner circumference of the frame 104 as previously illustrated for the compliant actuators 116 and the noncompliant actuators 120. The compliant actuators 180 can be mounted to the frame alone or together with either or both of the compliant actuators 116 and the noncompliant actuators 120.

In the example illustrated in FIG. 9, the compliant actuator 180 includes a proximal connector hub 184, a distal connector base 188, and an elastic element, such as a spring member 192, and a locking head 196. The spring member 192 has a distal end attached to the distal connector base 188 and a proximal end attached to the locking head 196. A pull member 200, such as a cable or rod, can be releasably coupled to the locking head 196 (for example, by a threaded connection) and used to displace the locking head 196 in the proximal direction. For example, the distal end portion of the pull member 200 can include a male threaded member that is configured to be tightened into a female threaded bore of the locking head 196, or the distal end portion of the pull member 200 can include a female threaded bore configured to receive a male threaded member of the locking head 196. The pull member 200 can be a component of a delivery apparatus and can be actuated by a control member (for example, a rotatable knob) on the handle of the delivery apparatus, as further described below.

The proximal connector hub 184 includes an opening 204, which can receive the locking head 196 and through which the pull member 200 can extend. In addition, the proximal connector hub 184 includes a lock arrangement within the opening 204 that can engage with a complementary lock arrangement on the locking head 196. In the radially expanded state of the frame 104, the locking head 196 is retained within the opening 204 via the lock arrangements. In the radially compressed state of the frame 104, the locking head 196 is located between the proximal connector hub 184 and the distal connector base 188. The pull member 200 can be released after docking the locking head 196 in the proximal connector hub 184.

The proximal connector hub 184 and the distal connector base 188 can include pin members 186, 190, respectively, or other suitable fasteners for mounting the proximal connector hub 184 and the distal connector base 188 to the frame 104, as illustrated in FIG. 10. In some examples, the pin members 186, 190 can form part of pivot joints 148 in the frame 104, as described for the pin members 158, 162 of the proximal connector head 156 and the distal connector base 160 of the compliant actuator 116.

While the distal connector base 188 is shown in FIGS. 9 and 10 to have similar dimensions to the proximal connector hub 184, it is to be understood that the distal connector base 188 can be much smaller than the proximal connector hub 184. In some cases, the distal connector base 188 can be a narrow structure (for example, a pin) that is rigidly attached to the distal end of the spring member 192 and that can be mounted on the frame (for example, forming part of a pivot joint in the frame).

In one example, the spring member 192 is an extension spring (or tension spring), which means that the spring member 192 is compressed (has an initial tension) in an unloaded position (or free state) and extends (that is, stretches) in a loaded position (that is, when an outside force larger than the initial tension is applied to the spring). In one example, the spring member 192 can be a helical or coil extension spring made of a biocompatible metal or alloy, such as stainless steel.

Unlike the compliant actuator 116 previously described where the spring member 164 is in a tensioned state when the frame 104 is radially compressed, the compliant actuator 180 can be configured such that the spring member 192 is in a free state when the frame 104 is radially compressed (or when the prosthetic heart valve is in a compressed or crimped state). As shown in FIG. 10, the locking head 196 is between the proximal connector hub 184 and the distal connector base 188 when the spring member 192 is in the free state and the prosthetic heart valve is in the compressed or crimped state.

The frame 104 with compliant actuators 180 can be radially compressed by pivoting or bending the struts of the frame in a direction that radially compresses the frame as previously described. For delivery of the prosthetic heart valve to the implantation site in a crimped state, the frame 104 can be retained in the radially compressed state using a loop member or capsule or another restraining device. At the implantation site, the compliant actuators 180 can be actuated to radially expand the frame 104 and lock the frame 104 in the radially expanded state.

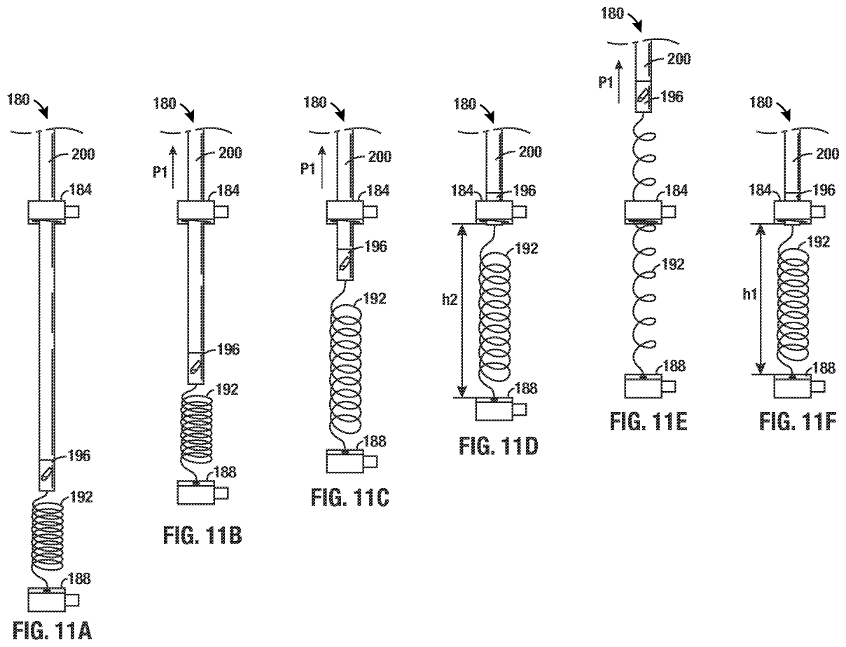

FIGS. 11A-11D illustrate different stages of actuation with the compliant actuator 180. While the description below proceeds with reference to a single compliant actuator 180, it should be noted that a prosthetic valve can include multiple compliant actuators 180 which are actuated in the same way. In FIG. 11A, the spring member 192 is in the free state, and the locking head 196 is between the distal connector base 188 and the proximal connector hub 184. In FIG. 11B, tension or a pulling force is being applied to the pull member 200 in a proximal direction, pulling the locking head 196, the spring member 192, and the distal connector base 188 in the proximal direction, causing the frame 104 to radially expand. The transition from FIG. 11A to FIG. 11B is along expansion diameters of the prosthetic heart valve prior to the prosthetic heart valve contacting the surrounding anatomy. During this phase, the spring member 192 can remain in a free state.

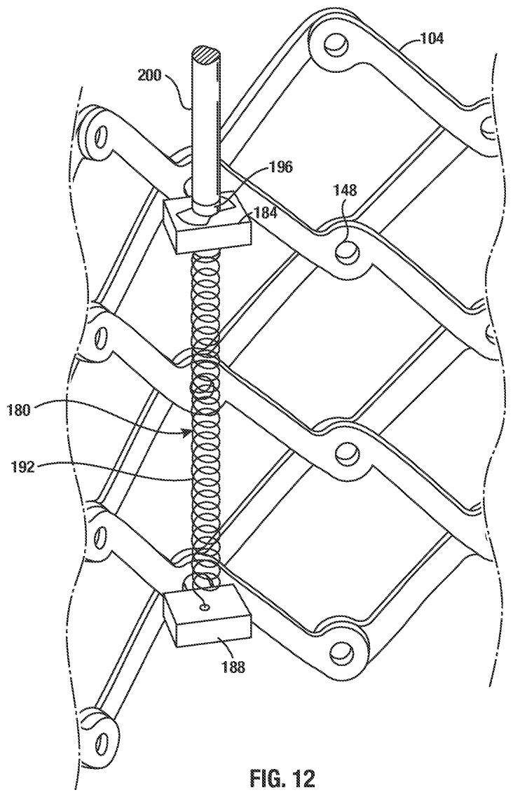

When the prosthetic heart valve 100 begins to press against the surrounding anatomy, the surrounding anatomy applies a resistive counter-force, causing the spring member 192 to extend (stretch) as the pull member 200 is further pulled in the proximal direction, as shown in FIG. 11C. In one example, the pull member 200 can be pulled proximally until the locking head 196 is positioned within the proximal connector hub 184 and retained in the proximal connector hub 184, as shown in FIGS. 11D and 12. The spring constant of the spring member 192 can be selected such that the outward radial force of the frame 104 produced by the spring member can be in equilibrium with the counter-force applied by the surrounding anatomy for the working range of the prosthetic valve (for example, between 26 mm and 29 mm, as one example).

In another example, the pull member 200 can be further pulled in the proximal direction such that the spring member 192 is further extended (stretched), as shown in FIG. 11E. This further extension contributes additional linear actuation to over-expand the prosthetic valve. The over-expansion of the prosthetic valve serves to partially expand the annulus the native valve so that when the spring member 192 is released and the locking head 196 is retained in the proximal connector hub 184, as shown in FIG. 11F, a new equilibrium is reached in which the native annulus and the prosthetic heart valve are expanded to a wider diameter. For example, the axial distance h1 between the distal connector base 188 and proximal connector hub 184 is smaller after over-expansion (shown in FIG. 11F) compared to the axial distance h2 between the distal connector base and proximal connector hub 184 without over-expansion (shown in FIG. 11D). The smaller axial distance h1 indicates a wider diameter of the frame. Conversely, the larger axial distance h2 indicates a narrower diameter of the frame.

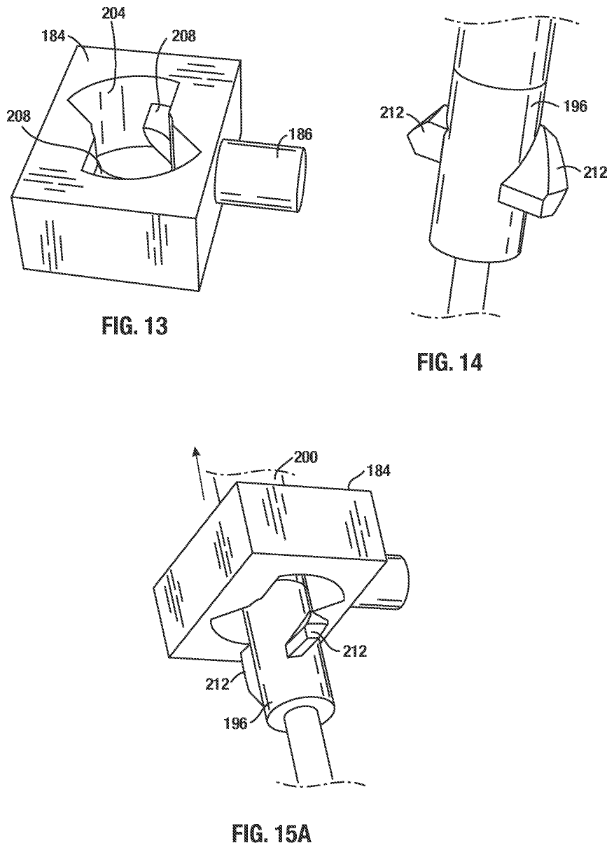

In one example, the locking head 196 is retained in the proximal connector hub 184 using a Bayonet-type lock. As illustrated in FIG. 13, the proximal connector hub 184 includes shoulders 208 projecting into the opening 204. The shoulders 208 can be positioned along a spiral path. As illustrated in FIG. 14, the locking head 196 includes protrusions 212 that are complementary to the shoulders 208. The protrusions 212 can be positioned along a spiral path.

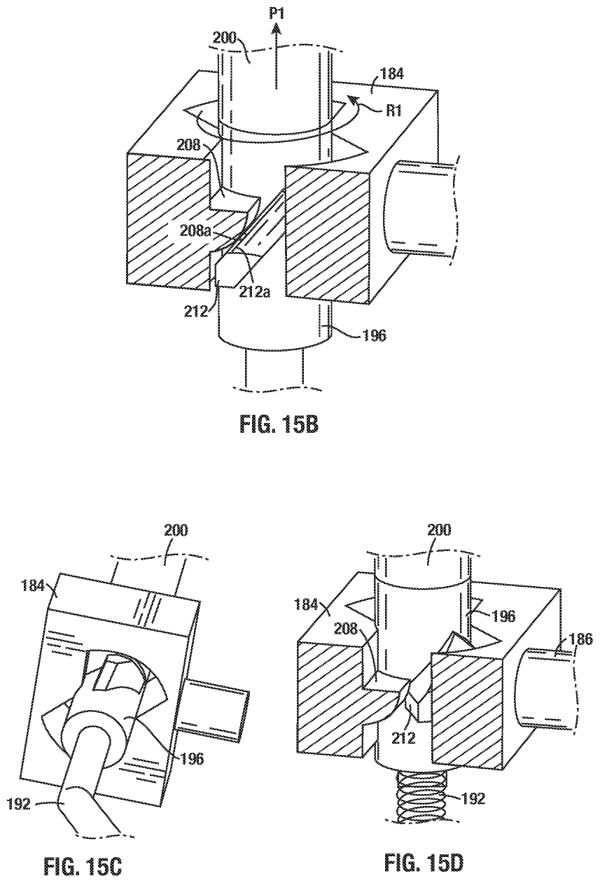

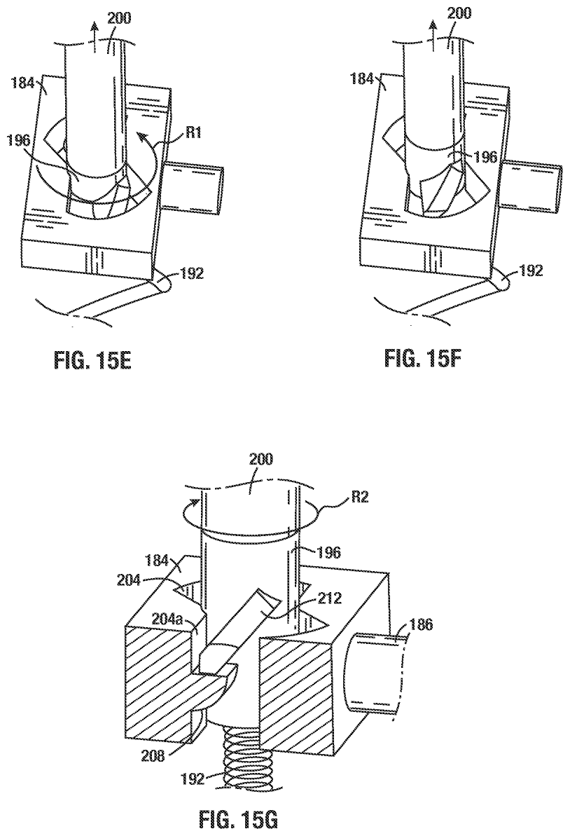

When the pull member 200 is pulled proximally, as shown in FIG. 15A, the locking head 196 is displaced toward the proximal connector hub 184 until the protrusions 212 contact the shoulders 208, as shown in FIG. 15B. The shoulders 208 have lower (or distal) angled surfaces 208a facing the protrusions 212, and the protrusions 212 have complementary upper (or proximal) angled surfaces 212a. The angled surfaces 208a, 212a are configured to allow the protrusions 212 to slide over the shoulders 208 when the locking head 196 is pulled in a proximal direction P1 in a manner that causes the locking head 196 to rotate in a first rotational direction R1. FIGS. 15B-15F show various positions of the locking head 196 during the sliding and rotational movement.

The locking head 196 can include a torsion spring that is biased in a second rotational direction (shown as R2 in FIG. 15G) when the protrusions 212 slide over the shoulders 208 as shown in FIGS. 15F and 15G. Thus, in the position shown in FIG. 15G, the locking head 196 rotationally springs back in the second rotational direction until the protrusions 212 are forcibly pressed against vertical flat surfaces 204a of the opening 204 of the proximal connector hub 184 above the shoulders 208. In some cases, the spring member 192 can serve as the torsion spring for angularly biasing the locking head 196.

The locking head 196 is rotated to allow the angled surfaces 212a to slide over the angled surfaces 208a of the shoulders 208 and to allow the protrusions 212 to be positioned above the shoulders 208, as shown in FIGS. 15E and 15F. The locking head 196 can engage the surfaces 204a of the proximal connector hub 184 and be thereby retained within the proximal connector hub 184 and prevent the locking head 196 from being pulled through the proximal connector hub 184 in the distal direction under the force of the spring member 192. It is possible to displace the locking head 196 proximally from the proximal connector hub 184 by continued pulling of the pull member 200 (for example, in order to over-expand the prosthetic valve, if desired). For example, the locking head 196 can be released from the vertical walls 204a by rotation and pulled proximally from the proximal connector hub 184.

The compliant actuator 180 can be configured to exert radial force against the anatomy for a range of annulus sizes with some safety, reaching an equilibrium with the surrounding anatomy. Moreover, if the native annulus dilates over time, the force of the compliant actuator 180 can cause the frame 104 to further expand and maintain equilibrium with the native annulus. This can be contrasted with, for example, a ratcheting mechanism that will exert radial force against the anatomy that depends only on the locked state of the ratchet and not the anatomical counter-force. The compliant actuator 180 can significantly improve the crimp profile of the prosthetic heart valve since the actuator relies on relatively small-sized or thin components such as wire-based spring.

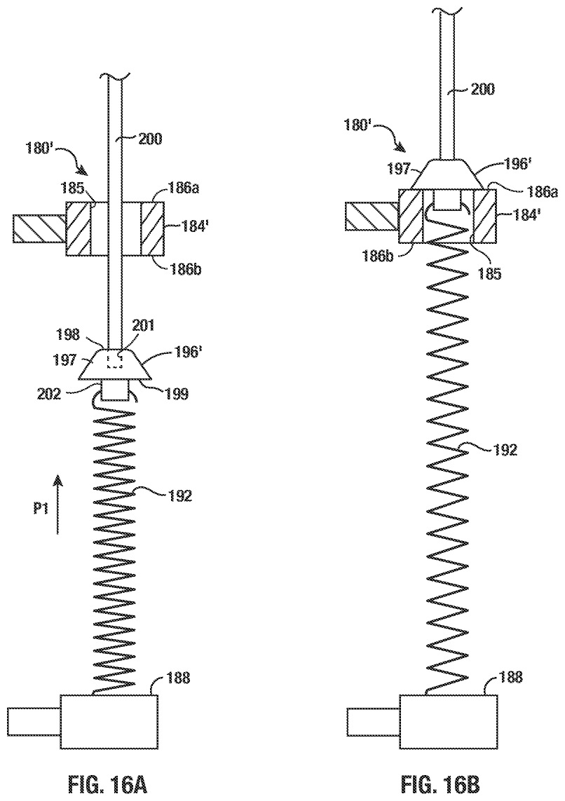

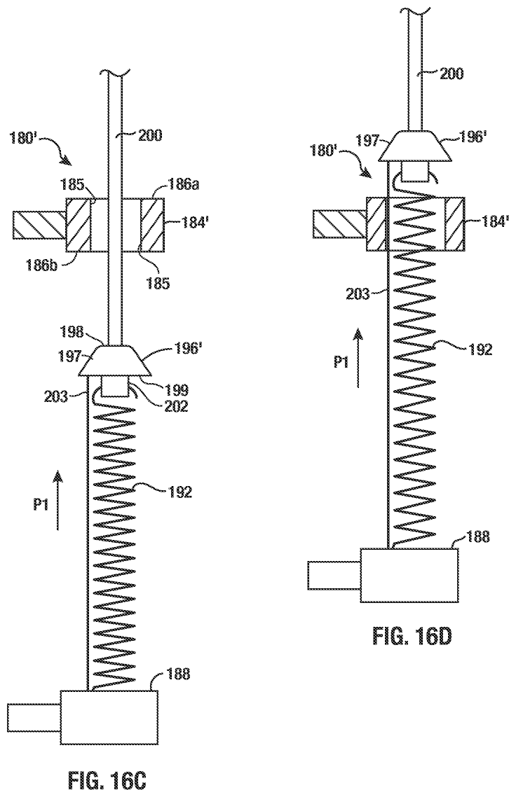

FIGS. 16A and 16B illustrate a compliant actuator 180′ with a snap-fit lock arrangement between a locking head 196′ and a proximal connector hub 184′, according to another example. The compliant actuator 180′ operates similarly to the previously described compliant actuator 180, with the exception of the particular manner in which the locking head 196′ is retained on the proximal connector hub 184′ when the frame 104 is in the radially expanded state.

The locking head 196′ includes a frustoconical head portion 197 (or a tapered head portion) having a proximal base 198 and a distal base 199. The diameter of the locking head 196′ at the proximal base 198 is smaller than the diameter of the locking head 196′ at the distal base 199. The distal base 199 can include a protrusion 202 extending in the distal direction. The proximal end of the spring member 192 can be coupled to the protrusion 202 (for example, the protrusion 202 can have a hole that receives a hook at the proximal end of the spring member 192). A hole 201 can be formed within the frustoconical head portion 197 to receive a distal end portion of the pull member 200. In one example, the hole 201 and the end portion of the pull member 200 can include complementary threads to form a threaded connection, which can releasably couple the pull member 200 to the locking head 196′.

The proximal connector hub 184′ includes a central opening 185, which can be a straight hole (as shown) or can have a different shape (for example, a tapered hole). The locking head 196′ is configured for passage through the central opening 185. A pull force can be applied to the pull member 200 to displace the locking head 196′ relative to the proximal connector 184′ in the proximal direction P1. The pull force can be effective in pulling the locking head 196 through the central opening 185 to a position proximal to the proximal connector hub 184′. The diameter of the proximal base 198 of the frustoconical head portion 197 can be smaller than the diameter of the central opening 185 at the distal end 186b of the proximal connector hub 184′ to facilitate entry of the locking head 196′ into the central opening 185′. The diameter of the distal base 199 of the frustoconical head portion 197 can be larger than the diameter of the central opening 185 at the proximal end 186a of the proximal connector hub 184′ to prevent movement of the locking head 196′ in a distal direction, as further described below. The locking head 196′ (or the frustoconical head portion 197) can be made of a flexible material to allow the locking head 196 to squeeze through the central opening at the proximal end 186a.

A pull force can be applied to the pull member 200 to displace the locking head 196′ in a proximal direction. The pull force can pull the locking head 196′ through the central opening 185 and to a location proximal to the proximal connector hub 184′. When the pull force is released from the pull member 200, the locking head 196′ will move in the distal direction (opposite to the proximal direction P1) by action of the spring member 192. Since the diameter of the distal base 199 of the frustoconical head portion 197 of the locking head 196′ is larger than the diameter of the central opening 185 at the proximal end 186a, the proximal end 186a will act as a stopping surface for the locking head 196′, preventing the frustoconical head portion 197 from sliding back into central opening 185. Thus, the locking head 196′ is retained on the proximal connector hub 184′ by the distal base 199 of the frustoconical head portion 197 that abuts the proximal end 186a of the proximal connector hub 184′ and by the action of the spring member 192 that urges the distal base 199 against the proximal end 186a.

In some cases, when the locking head 196′ is being displaced in a proximal direction by applying a pull force to the pull member 200, the surrounding anatomy may apply a resistive force that is higher than the maximum elongation of the spring member 192, which can prevent the locking head 196′ from being positioned proximal to the proximal connector hub 184′ and prevent locking of the frame 104 in the radially expanded state. To avoid this scenario, a tension member 203 (for example, a wire or string) can be attached between the distal connector base 188 and the locking head 196′, as illustrated in FIG. 16C. When the tension member 203 is tensioned to its maximum length by the pull force, the tension member 203 forms a rigid link between the locking head 196′ and the distal connector base 188 such that further application of the pull force by the pull member 200 pulls the tension member 203 and the distal connector base 188 in unison, as shown in FIG. 16D. The tension member 203 can also be incorporated in the actuator 180 of FIGS. 9-15.

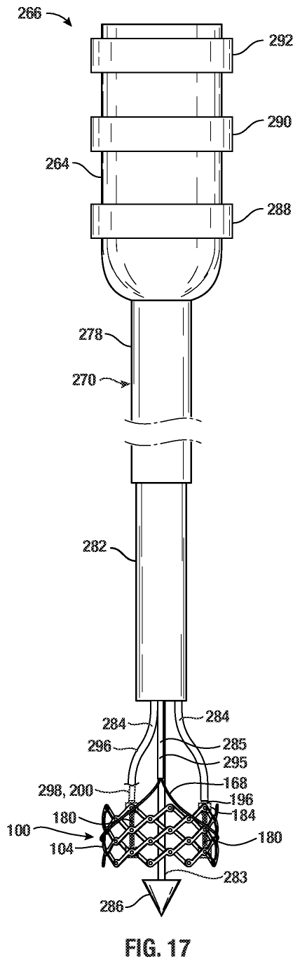

FIG. 17 shows an exemplary delivery assembly 266 including a delivery apparatus 270 and the prosthetic heart valve 100. For ease of illustration, some details of the prosthetic heart valve 100 are omitted from FIG. 17. For example, only a portion of the frame 104 is shown, and the valvular structure 108 is not shown. In the example of FIG. 17, the prosthetic heart valve 100 includes a plurality of compliant actuators 180 (two compliant actuators 180 are shown for illustrative purposes). The prosthetic heart valve 100 is shown in the radially expanded state in FIG. 17 (corresponding to the state of the compliant actuators 180 illustrated in FIG. 11D). The prosthetic heart valve 100 can be positioned in a crimped state (corresponding to the state of the compliant actuators 180 illustrated in FIG. 11A) prior to delivery to an implantation site within a patient's body.

The delivery apparatus 270 can generally include a handle 264, a first shaft 278 (an outer shaft in the illustrated example) extending distally from the handle 264, a second shaft 282 (an intermediate shaft in the illustrated example) extending through the first shaft 278, and a third shaft 283 (an inner shaft in the illustrated example) extending through the second shaft 282. In some cases, the second shaft 282 can be a multi-lumen shaft. The prosthetic heart valve 100 can be disposed around a distal portion of the third shaft 283. A nosecone 286 can be mounted to the distal end of the third shaft 283. The third shaft 283 and the nosecone 286 can define a guidewire lumen sized for receiving a guidewire so that the delivery apparatus 270 can be advanced over a guidewire previously inserted into a patient's body.

In some cases, one or more actuation assemblies 284 can extend through lumens of the second shaft 282 or can be otherwise coupled to the second shaft 282. The prosthetic heart valve 100 can be releasably coupled to the second shaft 282, for example, via the actuation assemblies 284. In some cases, the actuation assemblies 284 can operate the compliant actuators 180. For example, each actuation assembly 284 can include an outer member 296 and an inner member 298 extending through the outer member 296. The inner member 298 of each actuation assembly 284 can be connected to the pull member 200 (or can be the pull member 200) of a respective compliant actuator 180. In this manner, axial movement of the inner member 298 relative to the outer member 296 is transferred to the locking head 196 of the compliant actuator 180 to produce radial expansion of the frame 104 or lock the frame 104 in the radially expanded state or unlock the frame 104 from a radially expanded state. The actuation assemblies 284 can be disengaged from the compliant actuators 180 after the prosthetic heart valve 100 has been deployed and secured against the native annulus.

In some cases, the delivery apparatus 270 can include a valve restraining device 285 configured to retain the prosthetic heart valve 100 in a radially compressed state during delivery through a patient's vasculature and control expansion of the prosthetic valve at or near the target implantation site. The valve restraining device 285 can have similar characteristics to the previously described valve restraining device 235. For example, the valve restraining device 285 can comprise a shaft 295 and the adjustable loop member 168 extending from the distal end of the shaft 295. The shaft 295 can have a proximal end connected to the handle 264 and can extend distally from the handle through the second shaft 282. The loop member 168 can be a loop formed along a distal end portion of an elongated tether that extends through the shaft 295. The free ends of the tether can be operatively coupled to the handle 264. The size of the loop member 168 encircling the prosthetic heart valve 100 can be adjusted by relative movement between the shaft 295 and the tether, as previously described for the delivery apparatus 220.

The handle 264 of the delivery apparatus 270 can include one or more control mechanisms (for example, knobs or other actuation mechanisms) for controlling different components of the delivery apparatus 270 to steer the delivery apparatus and/or to expand and/or deploy the prosthetic heart valve 100 at an implantation site. For example, as illustrated in FIG. 17, the handle 264 can include first, second, and third knobs 288, 290, and 292.

The first knob 288 can have the same characteristics described for the first knob 238 of the delivery apparatus 220. For example, the first knob 288 can be a rotatable knob configured as a steering mechanism that can adjust the curvature of the first shaft 278, as described for the delivery apparatus 220. Similarly, the second knob 290 can have the same characteristics described for the second knob 240 of the delivery apparatus 220. For example, the second knob 290 can be a rotatable knob configured to produce radial expansion and/or contraction of the prosthetic heart valve 100, as described for the delivery apparatus 220. In other examples, each of the knobs 288 and 290 can be actuated by sliding or moving the knob axially, such as pulling and/or pushing the knob.

The third knob 292 can be used to actuate the compliant actuators 180 of the prosthetic valve (when present) via the actuation assemblies 284. For example, rotation of the knob 292 in a first direction can move the inner members 298/pull members 200 proximally relative to the outer members 296, which in turn moves the locking head 196 proximally (as shown in FIGS. 11A-11C). Additional rotation of the knob 292 can be effective in docking the locking head 196 in the proximal connector hub 184 (as shown in FIG. 11D) and retaining the prosthetic heart valve in a radially expanded state. Further rotation of the knob 292 can be effective in lifting the locking head 196 from the proximal connector hub 184 to overexpand the prosthetic heart valve (as shown in FIG. 11E). Rotation of the knob 292 in a second direction can move the inner members 298/pull members 200 distally relative to the outer members 296 to return the locking head 196 to the proximal connector hub 184 (as shown in FIG. 11F). In other examples, the third knob 292 can be actuated by sliding or moving the third knob 292 axially, such as pulling and/or pushing the knob.

The second knob 290 and the third knob 292 can work cooperatively to recompress the prosthetic heart valve 100. For example, the prosthetic heart valve 100 can be compressed when the locking head 196 is not within the proximal connector hub 184. To recompress the prosthetic heart valve 100 (for example, from a radially expanded state), the locking head 196 can be undocked from the proximal connector hub 184 by rotation of the third knob 292 in the first direction as described above, which would unlock the frame 104 and allow recompression of the frame 104. While the frame 104 is unlocked, the second knob 290 can be rotated in a direction that results in compression of the frame 104 (for example, in a direction that results in tightening of the loop member 168 around the frame 104).

The delivery apparatus 270 can include a fourth knob (not shown) that can be operated to disconnect the actuation assemblies 284 from the locking heads 196 of the compliant actuators 180, thereby releasing the prosthetic heart valve 100 from the delivery apparatus 270. The fourth knob can be a rotatable knob operatively connected to the actuation assemblies 284 such that rotation of the fourth knob decouples the actuation assemblies 284 from the prosthetic heart valve 100. For example, rotation of the fourth knob can rotate the pull members 200 to unscrew them from the locking heads 196, effectively disconnecting the prosthetic valve from the delivery apparatus. In other examples, the fourth knob can be actuated by sliding or moving the fourth knob axially, such as pulling and/or pushing the knob.

To implant a prosthetic heart valve within the native aortic valve via a transfemoral delivery approach, the prosthetic heart valve is mounted in a radially compressed state along the distal end portion of a delivery apparatus. The prosthetic heart valve and the distal end portion of the delivery apparatus are inserted into a femoral artery and are advanced into and through the descending aorta, around the aortic arch, and through the ascending aorta. The prosthetic heart valve is positioned within the native aortic valve and radially expanded as previously described.

Alternatively, a prosthetic heart valve can be implanted within the native aortic valve in a transapical procedure, whereby the prosthetic heart valve (on the distal end portion of the delivery apparatus) is introduced into the left ventricle through a surgical opening in the chest and the apex of the heart and the prosthetic heart valve is positioned within the native aortic valve.

Alternatively, in a transaortic procedure, a prosthetic heart valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

To implant a prosthetic heart valve within the native mitral valve via a transseptal delivery approach, the prosthetic heart valve is mounted in a radially compressed state along the distal end portion of a delivery apparatus. The prosthetic heart valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve.

Alternatively, a prosthetic heart valve can be implanted within the native mitral valve in a transapical procedure, whereby the prosthetic heart valve (on the distal end portion of the delivery apparatus) is introduced into the left ventricle through a surgical opening in the chest and the apex of the heart and the prosthetic heart valve is positioned within the native mitral valve.

To implant a prosthetic heart valve within the native tricuspid valve, the prosthetic heart valve is mounted in a radially compressed state along the distal end portion of a delivery apparatus. The prosthetic heart valve and the distal end portion of the delivery apparatus are inserted into a femoral vein and are advanced into and through the inferior vena cava, and into the right atrium, and the prosthetic heart valve is positioned within the native tricuspid valve. A similar approach can be used for implanting the prosthetic heart valve within the native pulmonary valve or the pulmonary artery, except that the prosthetic heart valve is advanced through the native tricuspid valve into the right ventricle and toward the pulmonary valve/pulmonary artery.

Another delivery approach is a transatrial approach whereby a prosthetic heart valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic heart valve (on the distal end portion of the delivery apparatus) is inserted through an incision in the chest and an incision made through the wall of the right ventricle (typically at or near the base of the heart) for implanting the prosthetic heart valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic heart valves disclosed herein can be implanted using any of various delivery procedures and delivery devices known in the art.

Any of the systems, devices, apparatuses, etc. herein can be sterilized (e.g., with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

Additional examples based on principles described herein are enumerated below.

Example 1: A prosthetic heart valve comprises an annular frame comprising an inflow end, an outflow end, and a longitudinal axis extending from the inflow end to the outflow end and defining an axial direction, the annular frame movable between a radially expanded state and a radially compressed state. The prosthetic heart valve further comprises at least one compliant actuator comprising an extension spring coupled to the annular frame at first and second axially spaced locations. The extension spring is configured to resiliently urge the first and second locations toward each other to retain the annular frame in the radially expanded state against a radial compression force applied to the annular frame by tissue surrounding the prosthetic heart valve.

Example 2: A prosthetic heart valve according to any example herein, particularly Example 1, wherein the at least one compliant actuator further comprises a proximal connector head and a distal connector base coupled to the annular frame at the first and second axially spaced locations, wherein the extension spring extends between and is attached at opposite ends to the proximal connector head and the distal connector base.

Example 3: A prosthetic heart valve according to any example herein, particularly Example 2, wherein the annular frame comprises a plurality of interconnected struts defining a plurality of cells.

Example 4: A prosthetic heart valve according to any example herein, particularly Example 3, wherein the struts are interconnected by a plurality of pivot joints, and wherein each of the proximal connector head and the distal connector base is coupled to the annular frame at one of the pivot joints.

Example 5: A prosthetic heart valve according to any example herein, particularly Example 4, wherein the proximal connector head comprises a pin member forming a part of the pivot joint to which the proximal connector head is coupled, and wherein the distal connector base comprises a pin member forming a part of the pivot joint to which the distal connector base is coupled.

Example 6: A prosthetic heart valve according to any example herein, particularly any one of Examples 2 to 5, wherein the compliant actuator extends over at least one of the cells.

Example 7: A prosthetic heart valve according to any example herein, particularly any one of Examples 2 to 5, wherein the compliant actuator extends over two of the cells stacked in the axial direction.

Example 8: A prosthetic heart valve according to any example herein, particularly any one of Examples 2 to 7, wherein the compliant actuator further comprises at least one additional extension spring extending between and attached at opposite ends to the proximal connector head and the distal connector base.

Example 9: A prosthetic heart valve according to any example herein, particularly any one of Examples 2 to 8, can further comprise a tension member coupled to the distal connector base, wherein a force applied to the tension member displaces the distal connector base in a direction towards the proximal connector head.

Example 10: A prosthetic heart valve according to any example herein, particularly Example 9, wherein the tension member extends through an opening in the proximal connector head.

Example 11: A prosthetic heart valve according to any example herein, particularly any one of Examples 1 to 10, wherein the extension spring has an initial tension and a spring constant selected such that the extension spring applies a radial force to the annular frame in a range from 20N to 40N when the annular frame is retained in the radially expanded state.

Example 12: A prosthetic heart valve according to any example herein, particularly any one of Examples 1 to 11, wherein the annular frame has a working diameter in a range from 26 mm to 29 mm.

Example 13: A prosthetic heart valve according to any example herein, particularly any one of Examples 1 to 12, can further comprise a loop member extending around the annular frame and configured to apply a releasable restraining force to the annular frame.

Example 14: A prosthetic heart valve according to any example herein, particularly any one of Examples 1 to 13, can further comprise a valvular structure disposed within the annular frame, wherein the valvular structure comprises one or more leaflets that open and close to regulate blood flow through the prosthetic heart valve.

Example 15: A delivery assembly comprises a handle; at least one shaft extending distally from the handle; and a prosthetic heart valve according to any example herein, particularly any one of Examples 1 to 14, coupled to the at least one shaft.

Example 16: A prosthetic heart valve comprises an annular frame comprising an inflow end, an outflow end, and a longitudinal axis extending from the inflow end to the outflow end and defining an axial direction, the annular frame movable between a radially expanded state and a radially compressed state. The prosthetic heart valve further comprises a plurality of compliant actuators positioned at spaced locations along a circumference of the annular frame. Each of the compliant actuators comprises a proximal connector head, a distal connector base, and at least one extension spring. The proximal connector head and the distal connector base are coupled to the annular frame at axially spaced locations, and the at least one extension spring extends between and is attached at opposite ends to the proximal connector head and the distal connector base. The extension springs are configured to apply a radial expansion force to the annular frame.

Example 17: A prosthetic heart valve according to any example herein, particularly Example 16, wherein the compliant actuators are positioned at equally spaced apart locations along the circumference of the annular frame.

Example 18: A prosthetic heart valve according to any example herein, particularly Example 16 or Example 17, can further comprise a plurality of noncompliant actuators coupled to the annular frame, each noncompliant actuator comprising first and second members that are axially movable relative to each other to produce radial expansion and compression of the annular frame.

Example 19: A prosthetic heart valve according to any example herein, particularly Example 18, wherein the compliant actuators and noncompliant actuators are positioned in an alternating arrangement along the circumference of the annular frame.

Example 20: The prosthetic heart valve of any example herein, particularly any one of Examples 16 to 19, wherein the annular frame comprises a plurality of interconnected struts defining a plurality of cells.

Example 21: The prosthetic heart valve of any example herein, particularly Example 20, wherein the struts are interconnected by a plurality of pivot joints, and wherein each of the proximal connector heads and each of the distal connector bases is coupled to the annular frame at one of the pivot joints.

Example 22: A prosthetic heart valve according to any example herein, particularly Example 20 or Example 21, wherein each compliant actuator extends over at least one of the cells.

Example 23: A prosthetic heart valve according to any example herein, particularly any one of Examples 16 to 22, wherein each compliant actuator is configured to apply a radial force to the annular frame in a range from 20N to 40N when the annular frame is in the radially expanded state.

Example 24: A prosthetic heart valve according to any example herein, particularly any one of Examples 16 to 23, wherein the annular frame has a working diameter in a range from 26 mm to 29 mm.

Example 25: A prosthetic heart valve according to any example herein, particularly any one of Examples 16 to 24, can further comprise a loop member extending around the annular frame and configured to apply a releasable restraining force to the annular frame.

Example 26: A prosthetic heart valve according to any example herein, particularly any one of Examples 16 to 25, can further comprise a valvular structure disposed within and coupled to the annular frame, wherein the valvular structure comprises one or more leaflets.

Example 27: A delivery assembly comprises a handle; at least one shaft extending distally from the handle; and a prosthetic heart valve according to any example herein, particularly any one of Examples 16 to 26, coupled to the at least one shaft.

Example 28: A method comprises radially compressing the annular frame of the prosthetic heart valve according to any example herein, particularly any one of Examples 16 to 26, by applying tension to the extension springs of the compliant actuators of the prosthetic heart valve; applying a restraining force to the annular frame to maintain the annular frame in the radially compressed state; inserting the prosthetic heart valve with the annular frame in the radially compressed state into a body of a patient; advancing the prosthetic heart valve with the annular frame in the radially compressed state to an implantation site within the body of the patient; and radially expanding the annular frame by releasing the restraining force from the annular frame such that the extension springs revert to their respective free states; and retaining the annular frame in the radially expanded state by the compliant actuators.

Example 29: A prosthetic heart valve comprises an annular frame comprising an inflow end, an outflow end, and a longitudinal axis extending from the inflow end to the outflow end and defining an axial direction, the annular frame movable between a radially expanded state and a radially compressed state. The prosthetic heart valve further comprises a compliant actuator comprising a proximal connector hub and a distal connector base coupled to the annular frame at axially spaced locations, the proximal connector hub having a first lock arrangement; a locking head having a second lock arrangement complementary to the first lock arrangement; and a spring having a distal end attached to the distal connector base and a proximal end attached to the locking head. The locking head is movable axially relative to the proximal connector hub between a first position when the annular frame is in the radially compressed state and a second position when the annular frame is in the radially expanded state. When the locking head is moved to the second position, the second lock arrangement can engage the first lock arrangement to resist axial movement of the locking head relative to the proximal connector hub in a distal direction.

Example 30: A prosthetic heart valve according to any example herein, particularly Example 29, wherein the spring is configured to achieve a free state when the locking head is in the first position.

Example 31: A prosthetic heart valve according to any example herein, particularly Example 29 or Example 30, wherein the spring comprises a tension spring.

Example 32: A prosthetic heart valve according to any example herein, particularly any one of Examples 29 to 31, wherein the proximal connector hub comprises a central opening, and wherein the first lock arrangement is formed on an inner wall surface of the proximal connector hub defining the central opening.

Example 33: A prosthetic heart valve according to any example herein, particularly Example 32, wherein the first lock arrangement comprises a pair of shoulders having angled surfaces.

Example 34: A prosthetic heart valve according to any example herein, particularly Example 33, wherein the second lock arrangement is formed on an outer wall surface of the locking head and comprises a pair of protrusions having angled surfaces that are complementary to the angled surfaces of the pair of shoulders.

Example 35: A prosthetic heart valve according to any example herein, particularly Example 34, wherein the angled surfaces of the shoulders and protrusions are configured to allow the protrusions to slide over the shoulders in a proximal direction by axial displacement and rotational movement of the locking head.

Example 36: A prosthetic heart valve according to any example herein, particularly Example 35, wherein the inner wall surface of the proximal connector hub comprises flat wall surfaces positioned to abut the protrusions after the protrusions slide over the shoulders in the proximal direction.

Example 37: A prosthetic heart valve according to any example herein, particularly any one of Examples 33 to 36, wherein the shoulders are positioned along a spiral path on the inner wall surface of the proximal connector hub, and wherein the protrusions are positioned along a spiral path on the outer wall surface of the locking head.

Example 38: A prosthetic heart valve according to any example herein, particularly any one of Examples 29 to 37, can further comprise a pull member coupled to the locking head, wherein the pull member extends through the proximal connector hub when the locking head is positioned between the proximal connector hub and the distal connector base.

Example 39: A prosthetic heart valve according to any example herein, particularly any one of Examples 29 to 31, wherein the first lock arrangement comprises a proximal end of the proximal connector hub and a central opening formed within the proximal connector hub, wherein the second lock arrangement comprises a tapered body of the locking head, and wherein the tapered body is configured to pass through the central opening and snap onto the proximal end of the proximal connector hub by action of the spring.

Example 40: A prosthetic heart valve according to any example herein, particularly Example 39, can further comprise a tension member extending between and attached to the locking head and the distal connector base, wherein the tension member in a tensioned state forms a rigid link between the locking head and the distal connector base enabling the locking head and distal connector base to be pulled in a proximal direction.

Example 41: A prosthetic heart valve according to any example herein, particularly any one of Examples 29 to 40, further comprises a valvular structure disposed within the annular frame, wherein the valvular structure comprises one or more leaflets that open and close to regulate blood flow through the prosthetic heart valve.

Example 42: A delivery assembly comprises a handle; at least one shaft extending distally from the handle; and a prosthetic heart valve according to any example herein, particularly any one of Examples 29 to 41, coupled to the at least one shaft.

Example 43: A method comprises inserting the prosthetic heart valve according to any one of Examples 29 to 41 into a body of a patient, wherein the prosthetic heart valve is inserted with the annular frame in the radially compressed state and the spring in the free state; advancing the prosthetic heart valve to an implantation site within the body of the patient; axially displacing the locking head proximally relative to the distal connector base to extend the extension spring and position the locking head at the proximal connector hub; and retaining the locking head in the proximal connector hub by the first and second lock arrangements.

Example 44: The method of any example herein, particularly Example 43, further comprises, prior to retaining the locking head in the proximal connector hub, axially displacing the locking head proximally relative to the proximal connector hub to further extend the spring, wherein the further extension of the spring over-expands the prosthetic heart valve.

Example 45: A prosthetic heart valve comprises an annular frame comprising an inflow end, an outflow end, and a longitudinal axis extending from the inflow end to the outflow end and defining an axial direction, the annular frame movable between a radially expanded state and a radially compressed state. The prosthetic heart valve further comprises a plurality of compliant actuators positioned at spaced locations along a circumference of the annular frame. Each of the compliant actuators comprises a proximal connector hub and a distal connector base coupled to the annular frame at axially spaced locations, the proximal connector hub having a central opening and a first lock arrangement formed in the central opening; a locking head having a second lock arrangement complementary to the first lock arrangement; and a spring having a distal end attached to the distal connector base and a proximal end attached to the locking head. The locking head is movable from a first position between the proximal connector hub and the distal connector base in which the spring is in a relaxed state to a second position in which the second lock arrangement engages the first lock arrangement and the spring is in a loaded state.

Example 46: A prosthetic heart valve according to any example herein, particularly Example 45, wherein each compliant actuator further comprises a pull member releasably coupled to the locking head and slidable through the central opening.

Example 47: A prosthetic heart valve according to any example herein, particularly any one of Example 45 or Example 46, wherein the first lock arrangement comprises a pair of shoulders formed on an inner wall surface of the proximal connector hub defining the central opening.

Example 48: A prosthetic heart valve according to any example herein, particularly Example 47, wherein the second lock arrangement comprises a pair of protrusions formed on an outer wall surface of the locking head, the protrusions configured to contact and slide over the shoulders by axial displacement and rotational movement of the locking head.

Example 49: A prosthetic heart valve according to any example herein, particularly Example 48, wherein the shoulders are positioned along a spiral path on the inner wall surface of the proximal connector hub, and wherein the protrusions are positioned along a spiral path on the outer wall surface of the locking head.

Example 50: A prosthetic heart valve comprises an annular frame comprising an inflow end, an outflow end, and a longitudinal axis extending from the inflow end to the outflow end and defining an axial direction, the annular frame movable between a radially expanded state and a radially compressed state; at least one compliant actuator coupled to the annular frame, the at least one compliant actuator configured to produce radial expansion of the annular frame and retain the annular frame in the radially expanded state; and at least one noncompliant actuator coupled to the annular frame, the at least one noncompliant actuator configured to produce radial expansion and compression of the annular frame.

Example 51: A prosthetic heart valve according to any example herein, particularly Example 50, wherein the at least one compliant actuator comprises a proximal connector head, a distal connector base, and an extension spring, the proximal connector head and the distal connector base coupled to the annular frame at axially spaced locations, the extension spring extending between and attached at opposite ends to the proximal connector head and the distal connector base, wherein a free state of the extension spring retains the annular frame in the radially expanded state.

Example 52: A prosthetic heart valve according to any example herein, particularly Example 50, wherein the at least one compliant actuator comprises a proximal connector hub and a distal connector base coupled to the annular frame at axially spaced locations, the proximal connector hub having a first lock arrangement; a locking head having a second lock arrangement complementary to the first lock arrangement; and an extension spring having a distal end attached to the distal connector base and a proximal end attached to the locking head. The locking head is positioned between the proximal connector hub and the distal connector base in a free state of the extension spring.

Example 53: A prosthetic heart valve according to any example herein, particularly any one of Examples 50 to 52, wherein the at least one noncompliant actuator comprises first and second members that are axially movable relative to each other to produce the radial expansion and compression of the annular frame.

Example 54: A prosthetic heart valve according to any example herein, particularly any one of Examples 50 to 52, can further comprise a valvular structure disposed within the annular frame, wherein the valvular structure comprises one or more leaflets that open and close to regulate blood flow through the prosthetic heart valve.

Example 55: A method of implanting a prosthetic heart valve within an anatomy comprises positioning a prosthetic heart valve in a radially compressed state within the anatomy and radially expanding the prosthetic heart valve to a first diameter such that the prosthetic heart valve contacts the anatomy and a compliant actuator coupled to a frame of the prosthetic heart valve produces an expansion force that is in a first equilibrium with a counter-force applied to the frame by the anatomy.

Example 56: A method according to any example herein, particularly Example 55, wherein the compliant actuator comprises a connector hub and a connector base coupled to two axially spaced apart locations on the frame, and wherein radially expanding the prosthetic heart valve to the first diameter comprises axially displacing the two axially spaced apart locations relative to each other to decrease a distance between the two axially spaced locations.

Example 57: A method according to any example herein, particularly any one of Examples 55 to 56, wherein radially expanding the prosthetic heart valve to the first diameter comprises axially extending a spring member coupled to the connector hub and the connector base in a first direction to a first length.

Example 58: A method according to any example herein, particularly Example 57, wherein axially extending the spring member in the first direction to the first length positions a locking head coupled to the spring member at the connector hub.

Example 59: A method according to any example herein, particularly Example 58, further comprising engaging the locking head with the connector hub such that displacement of the locking head in a second direction opposite to the first direction is prevented.

Example 60: A method according to any example herein, particularly Example 59, wherein engaging the locking head with the connector hub comprises rotating the locking head relative to the connector hub to engage a surface of the locking head with a surface of the connector hub.

Example 61: A method according to any example herein, particularly any one of Examples 57 to 60, further comprising radially expanding the prosthetic heart valve from the first diameter to a second diameter, wherein radially expanding the prosthetic heart valve to the second diameter further comprises axially extending the spring member in the first direction to a second length greater than the first length.

Example 62: A method according to any example herein, particularly Example 61, wherein axially extending the spring member to the second length moves the locking head past the connector hub in the first direction.

Example 63: A method according to any example herein, particularly any one of Examples 61 to 62, wherein subsequent to radially expanding the prosthetic heart valve to the second diameter, allowing the spring member to contract back to the first length to position the locking head at the connector hub.

Example 64: A method according to any example herein, particularly any one of Examples 61 to 63, wherein the expansion force produced by the compliant actuator is in a second equilibrium with the counter-force applied to the frame by the anatomy when the prosthetic heart valve is expanded to the second diameter.

Example 65: A prosthetic heart valve comprises an annular frame comprising an inflow end, an outflow end, and a longitudinal axis extending from the inflow end to the outflow end and defining an axial direction, the annular frame movable between a radially expanded state and a radially compressed state; and at least one compliant actuator coupled to the annular frame, the at least one compliant actuator configured to produce radial expansion of the annular frame and retain the annular frame in the radially expanded state.

Example 66: A prosthetic heart valve according to any example herein, particularly Example 65, wherein the at least one compliant actuator comprises an extension spring coupled to the annular frame at first and second axially spaced locations, wherein the extension spring is configured to resiliently urge the first and second axially spaced locations toward each other to retain the annular frame in the radially expanded state against a radial compression force applied to the annular frame by tissue surrounding the prosthetic heart valve.

Example 67: A prosthetic heart valve according to any example herein, particularly Example 66, wherein the at least one compliant actuator further comprises a proximal connector head and a distal connector base coupled to the annular frame at the first and second axially spaced locations, wherein the extension spring extends between and is attached at opposite ends to the proximal connector head and the distal connector base.

Example 68: A prosthetic heart valve according to any example herein, particularly Example 67, wherein the annular frame comprises a plurality of interconnected struts defining a plurality of cells, wherein the struts are interconnected by a plurality of pivot joints, and wherein each of the proximal connector head and the distal connector base is coupled to the annular frame at one of the pivot joints.

Example 69: A prosthetic heart valve according to any example herein, particularly any one of Examples 66 to 68, further comprising a tension member coupled to the distal connector base, wherein a force applied to the tension member displaces the distal connector base in a direction towards the proximal connector head.

Example 70: A prosthetic heart valve according to any example herein, particularly Example 65, wherein the at least one compliant actuator comprises a proximal connector hub and a distal connector base coupled to the annular frame at axially spaced locations, the proximal connector hub having a first lock arrangement; a locking head having a second lock arrangement complementary to the first lock arrangement; and a spring having a distal end attached to the distal connector base and a proximal end attached to the locking head; wherein the locking head is movable axially relative to the proximal connector hub between a first position when the annular frame is in the radially compressed state and a second position when the annular frame is in the radially expanded state; and wherein when the locking head is moved to the second position, the second lock arrangement can engage the first lock arrangement to resist axial movement of the locking head relative to the proximal connector hub in a distal direction.

Example 71: A prosthetic heart valve according to any example herein, particularly Example 70, wherein the spring is configured to achieve a free state when the locking head is in the first position.

Example 72: A prosthetic heart valve according to any example herein, particularly any one of Examples 70 to 71, wherein the proximal connector hub comprises a central opening; wherein the first lock arrangement is formed on an inner wall surface of the proximal connector hub defining the central opening, and wherein the first lock arrangement comprises a pair of shoulders having angled surfaces; wherein the second lock arrangement is formed on an outer wall surface of the locking head and comprises a pair of protrusions having angled surfaces that are complementary to the angled surfaces of the pair of shoulders; and wherein the angled surfaces of the shoulders and protrusions are configured to allow the pair of protrusions to slide over the pair of shoulders in a proximal direction by axial displacement and rotational movement of the locking head.

Example 73: A prosthetic heart valve according to any example herein, particularly Example 72, wherein the shoulders are positioned along a spiral path on the inner wall surface of the proximal connector hub, and wherein the pair of protrusions are positioned along a spiral path on the outer wall surface of the locking head.

Example 74: A prosthetic heart valve according to any example herein, particularly any one of Examples 71 to 73, further comprising a pull member coupled to the locking head, wherein the pull member extends through the proximal connector hub when the locking head is positioned between the proximal connector hub and the distal connector base.

Example 75: A prosthetic heart valve according to any example herein, particularly any one of Examples 70 to 71, wherein the first lock arrangement comprises a proximal end of the proximal connector hub and a central opening formed within the proximal connector hub, wherein the second lock arrangement comprises a tapered body of the locking head, and wherein the tapered body is configured to pass through the central opening and snap onto the proximal end of the proximal connector hub by action of the spring.

Example 76: A prosthetic heart valve according to any example herein, particularly Example 75, further comprising a tension member extending between and attached to the locking head and the distal connector base, wherein the tension member in a tensioned state forms a rigid link between the locking head and the distal connector base enabling the locking head and distal connector base to be pulled in a proximal direction.

Example 77: A prosthetic heart valve according to any example herein, particularly any one of Examples 65 to 76, further comprising at least one noncompliant actuator coupled to the annular frame, the at least one noncompliant actuator configured to produce radial expansion and compression of the annular frame.

Example 78: A delivery assembly comprises a handle; at least one shaft extending distally from the handle; and a prosthetic heart valve coupled to the at least one shaft. The prosthetic heart valve comprises an annular frame movable between a radially expanded state and a radially compressed state; and at least one compliant actuator coupled to the annular frame, the at least one compliant actuator configured to produce radial expansion of the annular frame and retain the annular frame in the radially expanded state.

Example 79: A prosthetic heart valve according to any example herein, particularly, any of Examples 1-26, 29-41, 45-54, and 65-77, wherein the prosthetic heart valve is sterilized.

Example 80: A delivery assembly according to any example herein, particularly any of Examples 27 or 42 or 78, wherein the delivery assembly is sterilized.

In view of the many possible examples to which the principles of the disclosed invention may be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

1. A prosthetic heart valve comprising:
an annular frame comprising an inflow end, an outflow end, and a longitudinal axis extending from the inflow end to the outflow end and defining an axial direction, the annular frame movable between a radially expanded state and a radially compressed state; and
at least one compliant actuator coupled to the annular frame, the at least one compliant actuator configured to produce radial expansion of the annular frame and retain the annular frame in the radially expanded state.

2. The prosthetic heart valve of claim 1, wherein the at least one compliant actuator comprises an extension spring coupled to the annular frame at first and second axially spaced locations, wherein the extension spring is configured to resiliently urge the first and second axially spaced locations toward each other to retain the annular frame in the radially expanded state against a radial compression force applied to the annular frame by tissue surrounding the prosthetic heart valve.

3. The prosthetic heart valve of claim 2, wherein the at least one compliant actuator further comprises a proximal connector head and a distal connector base coupled to the annular frame at the first and second axially spaced locations, wherein the extension spring extends between and is attached at opposite ends to the proximal connector head and the distal connector base.

4. The prosthetic heart valve of claim 3, wherein the annular frame comprises a plurality of interconnected struts defining a plurality of cells, wherein the struts are interconnected by a plurality of pivot joints, and wherein each of the proximal connector head and the distal connector base is coupled to the annular frame at one of the pivot joints.

5. The prosthetic heart valve of claim 3, further comprising a tension member coupled to the distal connector base, wherein a force applied to the tension member displaces the distal connector base in a direction towards the proximal connector head.

6. The prosthetic heart valve of claim 1, wherein the at least one compliant actuator comprises:
a proximal connector hub and a distal connector base coupled to the annular frame at axially spaced locations, the proximal connector hub having a first lock arrangement;
a locking head having a second lock arrangement complementary to the first lock arrangement; and
a spring having a distal end attached to the distal connector base and a proximal end attached to the locking head;
wherein the locking head is movable axially relative to the proximal connector hub between a first position when the annular frame is in the radially compressed state and a second position when the annular frame is in the radially expanded state; and
wherein when the locking head is moved to the second position, the second lock arrangement can engage the first lock arrangement to resist axial movement of the locking head relative to the proximal connector hub in a distal direction.

7. The prosthetic heart valve of claim 6, wherein the spring is configured to achieve a free state when the locking head is in the first position.

8. The prosthetic heart valve of claim 6, wherein the proximal connector hub comprises a central opening;
wherein the first lock arrangement is formed on an inner wall surface of the proximal connector hub defining the central opening, and wherein the first lock arrangement comprises a pair of shoulders having angled surfaces;
wherein the second lock arrangement is formed on an outer wall surface of the locking head and comprises a pair of protrusions having angled surfaces that are complementary to the angled surfaces of the pair of shoulders; and
wherein the angled surfaces of the shoulders and protrusions are configured to allow the pair of protrusions to slide over the pair of shoulders in a proximal direction by axial displacement and rotational movement of the locking head.

9. The prosthetic heart valve of claim 8, wherein the shoulders are positioned along a spiral path on the inner wall surface of the proximal connector hub, and wherein the pair of protrusions are positioned along a spiral path on the outer wall surface of the locking head.

10. The prosthetic heart valve of claim 6, further comprising a pull member coupled to the locking head, wherein the pull member extends through the proximal connector hub when the locking head is positioned between the proximal connector hub and the distal connector base.

11. The prosthetic heart valve of claim 6, wherein the first lock arrangement comprises a proximal end of the proximal connector hub and a central opening formed within the proximal connector hub, wherein the second lock arrangement comprises a tapered body of the locking head, and wherein the tapered body is configured to pass through the central opening and snap onto the proximal end of the proximal connector hub by action of the spring.

12. The prosthetic heart valve of claim 11, further comprising a tension member extending between and attached to the locking head and the distal connector base, wherein the tension member in a tensioned state forms a rigid link between the locking head and the distal connector base enabling the locking head and distal connector base to be pulled in a proximal direction.

13. The prosthetic heart valve of claim 1, further comprising at least one noncompliant actuator coupled to the annular frame, the at least one noncompliant actuator configured to produce radial expansion and compression of the annular frame.

14. A delivery assembly comprising:
a handle;
at least one shaft extending distally from the handle; and
a prosthetic heart valve coupled to the at least one shaft, the prosthetic heart valve comprising:
an annular frame movable between a radially expanded state and a radially compressed state; and
at least one compliant actuator coupled to the annular frame, the at least one compliant actuator configured to produce radial expansion of the annular frame and retain the annular frame in the radially expanded state.

15. A method of implanting a prosthetic heart valve within an anatomy, the method comprising:
positioning a prosthetic heart valve in a radially compressed state within the anatomy; and
radially expanding the prosthetic heart valve to a first diameter such that the prosthetic heart valve contacts the anatomy and a compliant actuator coupled to a frame of the prosthetic heart valve produces an expansion force that is in a first equilibrium with a counter-force applied to the frame by the anatomy.

16. The method of claim 15, wherein the compliant actuator comprises a connector hub and a connector base coupled to two axially spaced apart locations on the frame, and wherein radially expanding the prosthetic heart valve to the first diameter comprises axially displacing the two axially spaced apart locations relative to each other to decrease a distance between the two axially spaced apart locations.

17. The method of claim 16, wherein radially expanding the prosthetic heart valve to the first diameter comprises axially extending a spring member coupled to the connector hub and the connector base in a first direction to a first length; and
wherein axially extending the spring member in the first direction to the first length positions a locking head coupled to the spring member at the connector hub.

18. The method of claim 17, further comprising engaging the locking head with the connector hub such that displacement of the locking head in a second direction opposite to the first direction is prevented.

19. The method of claim 18, wherein engaging the locking head with the connector hub comprises rotating the locking head relative to the connector hub to engage a surface of the locking head with a surface of the connector hub.

20. The method of claim 17, further comprising:
radially expanding the prosthetic heart valve from the first diameter to a second diameter, wherein radially expanding the prosthetic heart valve to the second diameter further comprises axially extending the spring member in the first direction to a second length greater than the first length.
